(19) United States
(12) Patent Application Publication
(10) Pub. No.: US 2024/0122186 A1
(43) Pub. Date: Apr. 18, 2024

(54) METHYLOBACTERIUM STRAINS FOR CONTROLLING CORN ROOTWORM

(71) Applicant: NewLeaf Symbiotics, Inc., St. Louis, MO (US)

(72) Inventors: Marcus Jones, Ballwin, MO (US); Gregg Bogosian, Clarkson Valley, MO (US); Allison Jack, St. Louis, MO (US); Dayna Collett, St. Louis, MO (US)

(73) Assignee: NewLeaf Symbiotics, Inc., St. Louis, MO (US)

(21) Appl. No.: 18/488,471

(22) Filed: Oct. 17, 2023

Related U.S. Application Data
(60) Provisional application No. 63/379,797, filed on Oct. 17, 2022.

(51) Int. Cl. A01N 63/20; A01N 47/44; A01P 7/04; C12N 1/20

(52) U.S. Cl. CPC A01N 63/20; A01N 47/44; A01P 7/04; C12N 1/205; C12R 2001/01

(57) ABSTRACT

The present disclosure provides compositions comprising Corn Rootworm (CRW)-active Methylobacterium sp., methods for controlling CRW, and methods of making the compositions. Also provided are isolated CRW-active Methylobacterium sp.

### CROSS-REFERENCE TO RELATED APPLICATIONS

This patent application claims the benefit of U.S. Provisional Patent Application No. 63/379,797, filed Oct. 17, 2022, which is incorporated herein by reference in its entirety.

### BACKGROUND

One-carbon organic compounds such as methane and methanol are found extensively in nature, and are utilized as carbon sources by bacteria classified as methanotrophs and methylotrophs. Methanotrophic bacteria include species in the genera Methylobacter, Methylomonas, Methylomicrobium, Methylococcus, Methylosinus, Methylocystis, Methylosphaera, Methylocaldum, and Methylocella (Lidstrom, 2006). Methanotrophs possess the enzyme methane monooxygenase, that incorporates an atom of oxygen from 02 into methane, forming methanol. All methanotrophs are obligate one-carbon utilizers that are unable to use compounds containing carbon-carbon bonds. Methylotrophs, on the other hand, can also utilize more complex organic compounds, such as organic acids, higher alcohols, sugars, and the like. Thus, methylotrophic bacteria are facultative methylotrophs. Methylotrophic bacteria include species in the genera Methylobacterium, Hyphomicrobium, Methylophilus, Methylobacillus, Methylophaga, Aminobacter, Methylorhabdus, Methylopila, Methylosulfonomonas, Marinosulfonomonas, Paracoccus, Xanthobacter, Ancylobacter (also known as Microcyclus), Thiobacillus, Rhodopseudomonas, Rhodobacter, Acetobacter, Bacillus, Mycobacterium, Arthobacter, and Nocardia (Lidstrom, 2006).

Most methylotrophic bacteria of the genus Methylobacterium are pink-pigmented. They are conventionally referred to as PPFM bacteria, being pink-pigmented facultative methylotrophs. Green (2005, 2006) identified twelve validated species in the genus Methylobacterium, specifically M. aminovorans, M chloromethanicum, M. dichloromethanicum, M. extorquens, M. fujisawaense, M mesophilicum, M organophilum, M radiotolerans, M rhodesianum, M. rhodinum, M thiocyanatum, and M zatmanii. However, M. nidulans is a nitrogen-fixing Methylobacterium that is not a PPFM (Sy et al., 2001). Methylobacterium are ubiquitous in nature, being found in soil, dust, fresh water, sediments, and leaf surfaces, as well as in industrial and clinical environments (Green, 2006).

### SUMMARY

Provided herein are isolated CRW-active Methylobacterium sp., compositions comprising CRW-active Methylobacterium sp., methods of using the compositions to control CRW damage to plants, plant parts, and plants derived therefrom, and methods of making the compositions. Such CRW-active Methylobacterium sp. are in certain instances referred to herein as simply “Methylobacterium” or as “PPFM” (pink-pigmented facultative methylotrophs). In certain embodiments, CRW-active Methylobacterium sp. can be distinguished from other CRW-inactive Methylobacterium by assaying the Methylobacterium sp. for in vitro inhibition of CRW feeding, for inhibition of CRW damage to plants grown in a controlled environment (i.e. a growth chamber or greenhouse), for inhibition of CRW damage to plants grown, for the presence of nucleic acid polymorphisms characteristic of the CRW-active Methylobacterium sp., and combinations thereof. In certain embodiments, the CRW-active Methylobacterium sp. has at least one polymorphic DNA element that is present in at least one CRW-active Methylobacterium sp. strain selected from the group consisting of ISO13, ISO08, NLSO210 and NLS0617. In certain embodiments, the CRW-active Methylobacterium sp. has at least one polymorphic DNA element that is present in at least one CRW-active Methylobacterium sp. strain selected from the group consisting of ISO13, ISO08, NLSO210 and NLS0617. In certain embodiments, the CRW-active Methylobacterium sp. is a Methylobacterium isolate selected from the group consisting of ISO13, ISO08, NLSO210 and NLS0617.

Methods for controlling corn rootworm (CRW) damage to a corn plant that comprise: (i) applying a composition comprising a CRW-active Methylobacterium sp. to a corn plant, a part thereof, or a corn seed; and, (ii) growing the corn plant or a corn plant from the corn seed in the presence of CRW, thereby controlling CRW damage to the corn plant or to the corn plant from the corn seed are provided herein. In certain embodiments of the methods, CRW damage sustained by any of the corn plants grown in the presence of the CRW is reduced in comparison to CRW damage sustained by a control corn plant grown in the presence of the CRW. In certain embodiments of the methods, the composition comprises a solid substance with adherent CRW-active Methylobacterium grown thereon or an emulsion having CRW-active Methylobacterium grown therein. In certain embodiments of the methods, the composition comprises the CRW-active Methylobacterium sp. at a titer of about 5×10⁸, 1×10⁹, or 1×10¹⁰ colony-forming units per gram of the solid substance to about 5×10¹³ colony-forming units of Methylobacterium per gram of the solid substance or at a titer of about 1×10⁶ CFU/mL to about 1×10⁹ CFU/mL for the emulsion. In certain embodiments of the methods, the CRW-active Methylobacterium sp. has at least one polymorphic DNA element that is present in at least one CRW-active Methylobacterium sp. strain selected from the group consisting of ISO13, IS008, NLS0210 and NLS0617. In certain embodiments of the methods, the CRW-active Methylobacterium sp. has at least one polymorphic DNA element that is present in at least one CRW-active Methylobacterium sp. strain selected from the group consisting of ISO13, IS008, NLS0210 and NLS0617. In certain embodiments of the methods, the CRW-active Methylobacterium sp. is selected from the group consisting of IS013, IS008, NLS0210 and NLS0617. In certain embodiments of any of the aforementioned methods, the applied composition coats or partially coats the corn plant, the part thereof, or the corn seed. In certain embodiments of the methods, the composition is applied to foliage of the corn plant. In certain embodiments of the aforementioned methods, the composition is applied to the corn seed. In certain embodiments of the methods, the composition is applied to the corn plant at about a vegetative emergence (VE), vegetative 1 (V1), vegetative 2 (V2), vegetative 3 (V3), vegetative 4 (V4), vegetative 5 (V5), or vegetative 6 (V6) stage. In certain embodiments of any of the aforementioned methods, the composition further comprises an insecticide that provides for inhibition of CRW growth and/or reductions in CRW-mediated plant damage. In certain embodiments where the composition further comprises an insecticide, the insecticide is selected from the group consisting of a pyrethrin, synthetic pyrethroid, oxadiazine, chloronicotinyl, neonicotinoid, nitroguanidine insecticide, triazole, organophosphate, pyrrol, pyrazole, diacylhydrazine, biological/fermentation product, and a carbamate.

Also provided are corn plants or corn plant part that is coated or partially coated with a composition comprising a CRW-active Methylobacterium sp. In certain embodiments, the corn plant or corn plant part is coated or partially coated with a composition that comprises a solid substance with adherent CRW— active Methylobacterium grown thereon or an emulsion comprising CRW-active Methylobacterium grown therein. In certain embodiments, the corn plant or corn plant part is coated or partially coated with a composition that comprises the CRW-active Methylobacterium sp. at a titer of about 5×10⁸, 1×10⁹, or 1×10¹⁰ colony-forming units per gram of the solid substance to about 5×10¹³ colony-forming units of Methylobacterium per gram of the solid substance or at a titer of about 1×10⁶ CFU/mL to about 1×10⁹ CFU/mL for the emulsion. In certain embodiments, the CRW-active Methylobacterium sp. in the composition has at least one polymorphic DNA element that is present in at least one CRW-active Methylobacterium sp. strain selected from the group consisting of ISO13, IS008, NLS0210 and NLS0617. In certain embodiments, the CRW-active Methylobacterium sp. in the composition has at least one polymorphic DNA element that is present in at least one CRW-active Methylobacterium sp. strain selected from the group consisting of ISO13, IS008, NLS0210 and NLS0617. In certain embodiments, the CRW-active Methylobacterium sp. is a Methylobacterium isolate selected from the group consisting of IS013, IS008, NLS0210 and NLS0617. In certain embodiments of any of the aforementioned corn plant parts, the corn plant part is selected from the group consisting of a coleoptile, leaf, a stalk, and a seed.

Also provided are methods for controlling corn rootworm (CRW) damage to a corn plant that comprise: (i) applying a composition comprising a CRW-active Methylobacterium sp. to soil where a corn plant is growing or will be grown, wherein the composition comprises a solid substance with adherent CRW— active Methylobacterium grown thereon or an emulsion having CRW-active Methylobacterium grown therein; and, (ii) growing a corn plant or a corn plant from corn seed in soil subjected to the application of the composition and in the presence of CRW. In certain embodiments of the methods, CRW damage sustained by the corn plant grown in the presence of the CRW is reduced in comparison to a control plant grown in the presence of the CRW. In certain embodiments of the methods, the composition comprises the CRW-active Methylobacterium sp. at a titer of about 5×10⁸, 1×10⁹, or 1×10¹⁰ colony-forming units per gram of the solid substance to about 5×10¹³ colony-forming units of Methylobacterium per gram of the solid substance or at a titer of about 1×10⁶ CFU/mL to about 1×10⁹ CFU/mL for the emulsion. In certain embodiments of the methods, the CRW-active Methylobacterium sp. has at least one polymorphic DNA element that is present in at least one CRW-active Methylobacterium sp. strain selected from the group consisting of ISO13, IS008, NLSO210 and NLS0617. In certain embodiments of the methods, the CRW-active Methylobacterium sp. has at least one polymorphic DNA element that is present in at least one CRW-active Methylobacterium sp. strain selected from the group consisting of ISO13, IS008, NLSO210 and NLS0617. In certain embodiments of the methods, the CRW-active Methylobacterium sp. is a Methylobacterium isolate selected from the group consisting of IS013, IS008, NLSO210 and NLS0617. In certain embodiments of any of the aforementioned methods, the composition is applied to the soil by broadcasting the composition, by drenching the soil with the composition, and/or by depositing the composition in furrow. In certain embodiments of the methods, the depositing in furrow is performed prior to placing corn seed in the furrow, at the same time as placing corn seed in the furrow, or after placing corn seed in the furrow. In certain embodiments of any of the aforementioned methods, the composition further comprises an insecticide that provides for inhibition of CRW growth and/or reductions in CRW-mediated plant damage. In certain embodiments where the composition further comprises an insecticide, the insecticide is selected from the group consisting of a pyrethrin, synthetic pyrethroid, oxadiazine, chloronicotinyl, neonicotinoid, nitroguanidine insecticide, triazole, organophosphate, pyrrol, pyrazole, diacylhydrazine, biological/fermentation product, and a carbamate.

Methods for treating a corn plant seed that can provide a corn rootworm (CRW) tolerant corn plant that comprises applying a composition comprising a CRW-active Methylobacterium sp. to a corn seed, thereby obtaining a treated seed that can provide a CRW tolerant corn plant are also provided. In certain embodiments of the methods, CRW damage sustained by the CRW tolerant corn plant grown from the treated seed and in the presence of the CRW is reduced in comparison to CRW damage sustained by a control corn plant grown from an untreated seed in the presence of CRW. In certain embodiments of the methods, the composition comprises a solid substance with adherent CRW-active Methylobacterium grown thereon or an emulsion having CRW-active Methylobacterium grown therein. In certain embodiments of the methods, the composition comprises the CRW-active Methylobacterium sp. at a titer of about 5×10⁸, 1×10⁹, or 1×10¹⁰ colony-forming units per gram of the solid substance to about 5×10¹³ colony-forming units of Methylobacterium per gram of the solid substance or at a titer of about 1×10⁶ CFU/mL to about 1×10⁹ CFU/mL for the emulsion. In certain embodiments of the methods, the CRW-active Methylobacterium sp. has at least one polymorphic DNA element that is present in at least one CRW-active Methylobacterium sp. strain selected from the group consisting of ISO13, ISO08, NLS0210 and NLS0617. In certain embodiments of the methods, the CRW-active Methylobacterium sp. has at least one polymorphic DNA element that is present in at least one CRW-active Methylobacterium sp. strain selected from the group consisting of ISO13, ISO08, NLSO210 and NLS0617. In certain embodiments of the methods, the CRW-active Methylobacterium sp. is a Methylobacterium isolate selected from the group consisting of ISO13, IS008, NLS0210 and NLS0617. In certain embodiments of any of the aforementioned methods, the applied composition coats or partially coats the corn seed. Also provided herein are treated corn seeds obtained by any of the aforementioned methods. In certain embodiments of any of the aforementioned methods, the composition further comprises an insecticide that provides for inhibition of CRW growth and/or reductions in CRW-mediated plant damage. In certain embodiments where the composition further comprises an insecticide, the insecticide is selected from the group consisting of a pyrethrin, synthetic pyrethroid, oxadiazine, chloronicotinyl, neonicotinoid, nitroguanidine insecticide, triazole, organophosphate, pyrrol, pyrazole, diacylhydrazine, biological/fermentation product, and a carbamate.

Also provided herein are methods for controlling corn rootworm (CRW) damage to a corn plant that comprise: (i) planting a corn seed that has been treated with a composition comprising a CRW-active Methylobacterium sp.; and, (ii) growing a CRW-tolerant corn plant from the treated corn seed in the presence of CRW. In certain embodiments of the methods, the CRW damage sustained by the CRW-tolerant corn plant grown in the presence of the CRW is reduced in comparison to CRW damage sustained by a control corn plant grown from untreated corn seed in the presence of CRW. In certain embodiments of the methods, the seed was treated with a composition that comprises a solid substance with adherent CRW-active Methylobacterium grown thereon or an emulsion having CRW-active Methylobacterium grown therein. In certain embodiments of the methods, the composition comprises the CRW-active Methylobacterium sp. at a titer of about 5×10⁸, 1×10⁹, or 1×10¹⁰ colony-forming units per gram of the solid substance to about 5×10¹³ colony-forming units of Methylobacterium per gram of the solid substance or at a titer of about 1×10⁶ CFU/mL to about 1×10⁹ CFU/mL for the emulsion. In certain embodiments of the methods, the CRW-active Methylobacterium sp. has at least one polymorphic DNA element that is present in at least one CRW-active Methylobacterium sp. strain selected from the group consisting of ISO13, IS008, NLS0210 and NLS0617. In certain embodiments of the methods, the CRW-active Methylobacterium sp. has at least one polymorphic DNA element that is present in at least one CRW-active Methylobacterium sp. strain selected from the group consisting of ISO13, IS008, NLSO210 and NLS0617. In certain embodiments of the methods, the CRW-active Methylobacterium sp. is a Methylobacterium isolate selected from the group consisting of IS013, IS008, NLSO210 and NLS0617. In certain embodiments of any of the aforementioned methods, the applied composition coats or partially coats the corn seed. In certain embodiments of any of the aforementioned methods, the composition further comprises an insecticide that provides for inhibition of CRW growth and/or reductions in CRW-mediated plant damage. In certain embodiments where the composition further comprises an insecticide, the insecticide is selected from the group consisting of a pyrethrin, synthetic pyrethroid, oxadiazine, chloronicotinyl, neonicotinoid, nitroguanidine insecticide, triazole, organophosphate, pyrrol, pyrazole, diacylhydrazine, biological/fermentation product, and a carbamate.

Also provided are compositions comprising a CRW-active Methylobacterium sp. and an agriculturally acceptable adjuvant and/or and agriculturally acceptable excipient. In certain embodiments, the composition comprises a solid substance with adherent CRW-active Methylobacterium grown thereon or an emulsion having CRW-active Methylobacterium grown therein. In certain embodiments, the composition comprises the CRW-active Methylobacterium sp. at a titer of about 5×10⁸, 1×10⁹, or 1×10¹⁰ colony-forming units per gram of the solid substance to about 5×10¹³ colony-forming units of Methylobacterium per gram of the solid substance or at a titer of about 1×10⁶ CFU/mL to about 1×10⁹ CFU/mL for the emulsion. In certain embodiments of the methods, the CRW-active Methylobacterium sp. has at least one polymorphic DNA element that is present in at least one CRW-active Methylobacterium sp. strain selected from the group consisting of ISO13, ISO08, NLS0210 and NLS0617. In certain embodiments, the CRW-active Methylobacterium sp. has at least one polymorphic DNA element that is present in at least one CRW-active Methylobacterium sp. strain selected from the group consisting of ISO13, ISO08, NLSO210 and NLS0617. In certain embodiments, the CRW-active Methylobacterium sp. is selected from the group consisting of ISO13, ISO08, NLSO210 and NLS0617. In certain embodiments, the composition further comprises an insecticide that provides for inhibition of CRW growth and/or reductions in CRW-mediated plant damage. In certain embodiments, the insecticide is selected from the group consisting of a pyrethrin, synthetic pyrethroid, oxadiazine, chloronicotinyl, neonicotinoid, nitroguanidine insecticide, triazole, organophosphate, pyrrol, pyrazole, diacylhydrazine, biological/fermentation product, and a carbamate.

Also provided herein is an isolated Methylobacterium sp. selected from the group consisting of NLSO210 and NLS0617.

### BRIEF DESCRIPTION OF THE DRAWINGS

The accompanying drawings, which are incorporated in and form a part of the specification, illustrate certain embodiments of the present disclosure. The patent or application file contains at least one drawing executed in color. Copies of this patent or patent application with color drawing(s) will be provided by the Office by request and payment of the necessary fee.

FIG. 1 compares lodging of untreated check plants (top) to plants treated with a CRW-active Methylobacterium at the V3 stage (bottom).

FIG. 2 compares CRW damage to roots of untreated check plants (top) to the roots of plants treated with a CRW-active Methylobacterium at the V3 stage (bottom).

### DESCRIPTION

### Definitions

As used herein, the phrases “adhered thereto” and “adherent” refer to Methylobacterium that are associated with a solid substance by growing, or having been grown, on a solid substance.

As used herein, the phrase “agriculturally acceptable adjuvant” refers to a substance that enhances the performance of an active agent in a composition for treatment of plants and/or plant parts. In certain compositions, an active agent can comprise a mono-culture or co-culture of Methylobacterium.

As used herein, the phrase “agriculturally acceptable excipient” refers to an essentially inert substance that can be used as a diluent and/or carrier for an active agent in a composition for treatment of plants and/or plant parts. In certain compositions, an active agent can comprise a mono-culture or co-culture of Methylobacterium.

As used herein, the term “Methylobacterium” refers to bacteria that are facultative methylotrophs of the genus Methylobacterium. The term Methylobacterium, as used herein, thus does not encompass includes species in the genera Methylobacter, Methylomonas, Methylomicrobium, Methylococcus, Methylosinus, Methylocystis, Methylosphaera, Methylocaldum, and Methylocella, which are obligate methanotrophs.

As used herein, the phrase “control plant” refers to a plant that had not received treatment with a CRW-active Methylobacterium or composition comprising the same at either the seed or any subsequent stage of the control plant's development. Control plants include, but are not limited to, non-transgenic plants, transgenic plants having a transgene-conferred CRW resistance trait, and plants treated with, or grown in soil treated with, an insecticidal compound or other agent that can protect a plant from CRW feeding.

As used herein, the terms “Corn Rootworm” and “CRW” are used interchangeable to refer to the larval or adult forms of any insect of the genus Diabrotica.

As used herein, the phrase “co-culture of Methylobacterium” refers to a Methylobacterium culture comprising at least two strains of Methylobacterium or at least two species of Methylobacterium.

As used herein, the phrase “contaminating microorganism” refers to microorganisms in a culture, fermentation broth, fermentation broth product, or composition that were not identified prior to introduction into the culture, fermentation broth, fermentation broth product, or composition.

As used herein, the term “emulsion” refers to a colloidal mixture of two immiscible liquids wherein one liquid is the continuous phase and the other liquid is the dispersed phase. In certain embodiments, the continuous phase is an aqueous liquid and the dispersed phase is liquid that is not miscible, or partially miscible, in the aqueous liquid.

As used herein, the phrase “essentially free of contaminating microorganisms” refers to a culture, fermentation broth, fermentation product, or composition where at least about 95% of the microorganisms present by amount or type in the culture, fermentation broth, fermentation product, or composition are the desired Methylobacterium or other desired microorganisms of pre-determined identity.

As used herein, the phrase “inanimate solid substance” refers to a substance which is insoluble or partially soluble in water or aqueous solutions and which is either non-living or which is not a part of a still-living organism from which it was derived.

As used herein, the phrase “mono-culture of Methylobacterium” refers to a Methylobacterium culture consisting of a single strain of Methylobacterium.

As used herein, the term “peptide” refers to any polypeptide of 50 amino acid residues or less.

As used herein, the term “protein” refers to any polypeptide having 51 or more amino acid residues.

As used herein, a “pesticide” refers to an agent that is insecticidal, fungicidal, nematocidal, bacteriocidal, or any combination thereof.

As used herein, the phrase “bacteriostatic agent” refers to agents that inhibit growth of bacteria but do not kill the bacteria.

As used herein, the phrase “pesticide does not substantially inhibit growth of said Methylobacterium” refers to any pesticide that when provided in a composition comprising a fermentation product comprising a solid substance wherein a mono-culture or co-culture of Methylobacterium is adhered thereto, results in no more than a 50% inhibition of Methylobacterium growth when the composition is applied to a plant or plant part in comparison to a composition lacking the pesticide. In certain embodiments, the pesticide results in no more than a 40%, 20%, 10%, 5%, or 1% inhibition of Methylobacterium growth when the composition is applied to a plant or plant part in comparison to a composition lacking the pesticide.

As used herein, the term “PPFM bacteria” refers without limitation to bacterial species in the genus Methylobacterium other than M nodulans.

As used herein, the phrase “solid substance” refers to a substance which is insoluble or partially soluble in water or aqueous solutions.

As used herein, the phrase “solid phase that can be suspended therein” refers to a solid substance that can be distributed throughout a liquid by agitation.

As used herein, the term “non-regenerable” refers to either a plant part or processed plant product that cannot be regenerated into a whole plant.

As used herein, the phrase “substantially all of the solid phase is suspended in the liquid phase” refers to media wherein at least 95%, 98%, or 99% of solid substance(s) comprising the solid phase are distributed throughout the liquid by agitation.

As used herein, the phrase “substantially all of the solid phase is not suspended in the liquid phase” refers to media where less than 5%, 2%, or 1% of the solid is in a particulate form that is distributed throughout the media by agitation.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

CRW-active Methylobacterium, compositions comprising CRW-active Methylobacterium, methods of their use, and methods of making

Various CRW-active Methylobacterium isolates, compositions comprising these Methylobacterium, methods of using the compositions to inhibit CRW growth and/or reduce CRW damage to a plant, and methods of making the compositions are provided herein. As used herein, inhibition of the growth of a CRW includes any measurable decrease in CRW growth, where CRW growth includes, but is not limited to, any measurable increase in the larval weight, and/or any progression through larval development of from larval to adult development. As used herein, inhibition of CRW growth and/or reduction of CRW damage to a plant are also understood to include any measurable decrease in CRW feeding and/or the adverse effects caused by CRW feeding on a plant. Adverse effects of CRW feeding on a plant include, but are not limited to, any type of tissue damage or necrosis, especially to nodal roots of a corn plant, increased incidence of fungal disease, any type of yield reduction, and/or increased lodging.

Isolated CRW-active Methylobacterium sp. are provided herein. In certain embodiments, the Methylobacterium is selected from the group consisting of M aminovorans, M. extorquens, M fujisawaense, M. mesophilicum, M radiotolerans, M. rhodesianum, M nodulans, M phyllosphaerae, M thiocyanatum, and M. oryzae. In certain embodiments, Methylobacterium is not M. radiotolerans or M. oryzae. In certain embodiments, the CRW-active Methylobacterium isolate is selected from the group consisting of IS013, IS014, IS008, NLSO210 and NLS0617. In certain embodiments, the CRW-active Methylobacterium isolate is selected from the group consisting of IS013, IS014, IS008, NLSO210 and NLS0617. In certain embodiments, the CRW-active Methylobacterium provides for at least about 25%, at least about 50%, or at least about 75% reductions in CRW damage to a treated plant, plant arising from a treated seed, or plant grown in soil treated with the CRW in comparison to untreated control plants, plants arising from untreated seeds, or plants grown in untreated soils upon exposure to a CRW. In certain embodiments, the CRW that is inhibited is selected from the group consisting of a Diabrotica balteata, Diabrotica barberi, Diabrotica undecimpunctata. and Diabrotica virgifera species.

In certain embodiments, the Methylobacterium is not M. radiotolerans or M oryzae. In certain embodiments, the CRW-active Methylobacterium provides for at least about 25%, at least about 50%, or at least about 75% reductions in CRW growth on a treated plant, plant arising from a treated seed, or plant grown in soil treated with the CRW in comparison to an untreated control plants, plants arising from untreated seeds, or plants grown in untreated soils upon exposure to a CRW. In certain embodiments, the CRW-active Methylobacterium is a Methylobacterium that inhibits a Diabrotica sp. is selected from the group consisting of a Diabrotica balteata, D. virgifera zea Krysan & Smith, Diabrotica barberi, Diabrotica undecimpunctata, and Diabrotica virgifera species. In certain embodiments of any of the aforementioned compositions, the composition comprises a solid substance wherein a mono-culture or co-culture of Methylobacterium is adhered thereto. In certain embodiments where the Methylobacterium is adhered to a solid substance, the composition comprises a colloid formed by the solid substance wherein a mono-culture or co-culture of Methylobacterium is adhered thereto and a liquid. In certain embodiments, the colloid is a gel. In certain embodiments of certain aforementioned compositions, composition is an emulsion that does not contain a solid substance. In certain embodiments of any of the aforementioned compositions, the CRW-active Methylobacterium is selected from the group consisting of IS013, ISO14, ISO08, NLSO210 and NLS0617. In certain embodiments of any of the aforementioned compositions, the CRW-active Methylobacterium is selected from the group consisting of IS013, ISO14, ISO08, NLSO210 and NLS0617.

In certain embodiments, isolated CRW-active Methylobacterium sp. can be identified by treating a plant, a seed, soil in which the plant or a plant arising from the seed are grown, or other plant growth media in which the plant or a plant arising from the seed are grown and assaying for either reductions in CRW damage, CRW growth, CRW feeding activity, and combinations thereof. In still other embodiments, the CRW-active Methylobacterium sp., compositions comprising the same, fermentation products comprising the same, cell free exudates therefrom, or compounds derived therefrom can be incorporated into a CRW diet that is fed to CRW larvae and thus assayed for inhibition of larval growth, development, or feeding activity. Various assays that can adapted for use in identifying CRW-active Methylobacterium sp. are disclosed in U.S. Pat. No. 8,080,496, U.S Patent Application Publication 20130116170, and U.S. Patent Publication No. 20120151634, which are each incorporated herein by reference in their entireties.

In certain embodiments, the CRW-active Methylobacterium sp. can be identified by testing newly isolated candidate Methylobacterium sp. for the presence of polymorphic nucleic acid sequences that are present in CRW-active Methylobacterium sp. provided herein and that are absent from Methylobacterium sp. provided herein that do not provide reductions in CRW damage when applied to a plant. In certain embodiments, the polymorphic nucleic acid sequences that are present in the identified CRW-active Methylobacterium sp. are also present in one or more of the CRW-active Methylobacterium sp. isolates ISO13, ISO14, ISO08, NLSO210 and NLS0617 provided herein that do not reduce CRW damage to a plant. In certain embodiments, the polymorphic nucleic acid sequences that are present in the identified CRW-active Methylobacterium sp. are also present in one or more of the CRW-active Methylobacterium sp. isolates ISO13, ISO14, ISO08, NLSO210 and NLS0617. In certain embodiments, the polymorphic nucleic acid sequences that are present in the identified CRW-active Methylobacterium sp. are also present in one or more of the CRW-active Methylobacterium sp. strains selected from the group consisting of ISO13, ISO14, ISO08, NLSO210 and NLS0617 provided herein, or any combination thereof. In certain embodiments, the polymorphic nucleic acid sequences that are present in the identified CRW-active Methylobacterium sp. are present in all of the CRW-active Methylobacterium sp. isolates ISO13, ISO14, ISO08, NLSO210 and NLS0617 provided herein. In certain embodiments, the polymorphic nucleic acid sequences that are present in the identified CRW-active Methylobacterium sp. are also present in all of the CRW-active Methylobacterium sp. isolates ISO13, ISO14, ISO08, NLSO210 and NLS0617. In certain embodiments, the polymorphic nucleic acid sequences that are present in the identified CRW-active Methylobacterium sp. are also present in one or more of the CRW-active Methylobacterium sp. isolates ISO13, ISO14, ISO08, NLSO210 and NLS0617. Such nucleic acid polymorphisms that occur in the CRW-active Methylobacterium sp. can include, but are not limited to, single nucleotide polymorphisms, RFLP, AFLP and/or other DNA variations such as repetitive sequences, insertion sequences, transposons, and genomic islands occurring as a result of insertions, deletions, and substitutions (Indels) in the bacterial genome which includes both the chromosomal DNA as well as any extrachromosomal nucleic acid elements that can be present in the CRW-active Methylobacterium sp. Such extrachromosomal nucleic acid elements include, but are not limited to, plasmids, bacteriophage DNA or RNA, and the like. Methods used to identify such nucleotide polymorphisms include, but are not limited to, single base extension (SBE) techniques, allele specific hybridization (ASH), real-time PCR detection (i.e. TaqMan™; U.S. Pat. Nos. 5,804,375; 5,538,848; 5,487,972; and 5,210,015, which are each incorporated herein by reference in their entireties), combinations of ASH and RT-PCR (KASP™ detection systems, LGC Genomics, Middlesex, UK) and deep sequencing techniques (U. S. Patent Appl. No. 20120264632, incorporated herein by reference in its entirety).

Various Methylobacterium sp. isolates provided herein are disclosed in Table 1.

TABLE 1
Methylobacterium sp. Isolates

| ISOLATE | NLS | USDA ARS | |
|---|---|---|---|
| No. | No. | NRRL No.¹ | Strain Source: Obtained from: |
| ISO01 | NLS0046 | NRRL B-50929 | a soybean plant grown in Saint Louis |
| | | | County, Missouri, USA |
| ISO02 | NLS0020 | NRRL B-50930 | a weed grown in Saint Louis County, |
| | | | Missouri, USA |
| ISO03 | NLS0017 | NRRL B-50931 | a mint plant grown in Saint Louis County, |
| | | | Missouri, USA |
| ISO04 | NLS0042 | NRRL B-50932 | a soybean plant grown in Saint Louis |
| | | | County, Missouri, USA |
| ISO05 | NLS0089 | NRRL B-50933 | a broccoli plant grown in Saint Louis |
| | | | County, Missouri, USA |
| ISO06 | NLS0068 | NRRL B-50934 | a corn plant grown in Saint Louis County, |
| | | | Missouri, USA |
| ISO07 | NLS0065 | NRRL B-50935 | a corn plant grown in Saint Louis County, |
| | | | Missouri, USA |
| ISO08 | NLS0069 | NRRL B-50936 | a corn plant grown in Saint Louis County, |
| | | | Missouri, USA |
| ISO09 | NLS0062 | NRRL B-50937 | a corn plant grown in Saint Louis County, |
| | | | Missouri, USA |
| ISO10 | NLS0064 | NRRL B-50938 | a corn plant grown in Saint Louis County, |
| | | | Missouri, USA |
| ISO11 | NLS0021 | NRRL B-50939 | a lettuce plant grown in Saint Louis County, |
| | | | Missouri, USA |
| ISO12 | NLS0066 | NRRL B-50940 | a corn plant grown in Saint Louis County, |
| | | | Missouri, USA |
| ISO13 | NLS0037 | NRRL B-50941 | a tomato plant grown in Saint Louis County, |
| | | | Missouri, USA |
| ISO14 | NLS0038 | NRRL B-50942 | a tomato plant grown in Saint Louis County, |
| | | | Missouri, USA |
| | NLS0210 | | a Japanese maple grown in Saint Louis |
| | | | County, Missouri, USA |
| | NLS0617 | | A giant sumpweed (Iva xanthifolia) growing |
| | | | in Woodbury County, Iowa, USA |

¹Deposit number for strain deposited with the AGRICULTURAL RESEARCH SERVICE CULTURE COLLECTION (NRRL) of the National Center for Agricultural Utilization Research, Agricultural Research Service, U.S. Department of Agriculture, 1815 North University Street, Peoria, Illinois 61604 U.S.A. under the terms of the Budapest Treaty on the International Recognition of the Deposit of Microorganisms for the Purposes of Patent Procedure. Subject to 37 CFR §1.808(b), all restrictions imposed by the depositor on the availability to the public of the deposited material will be irrevocably removed upon the granting of any patent from this patent application.

Samples of the following Methylobacterium sp. strains have been deposited with the AGRICULTURAL RESEARCH SERVICE CULTURE COLLECTION (NRRL) of the National Center for Agricultural Utilization Research, Agricultural Research Service, U.S. Department of Agriculture, 1815 North University Street, Peoria, Illinois 61604 U.S.A. under the terms of the Budapest Treaty on the International Recognition of the Deposit of Microorganisms for the Purposes of Patent Procedure. Methylobacterium sp. NRRL B-50929, NRRL B-50930, NRRL B-50931, NRRL B-50932, NRRL B-50933, NRRL B-50934, NRRL B-50935, NRRL B-50936, NRRL B-50937, NRRL B-50938, NRRL B-50939, NRRL B-50940, NRRL B-50941 and NRRL B-50942 were deposited with NRRL on Mar. 12, 2014.

Also provided herein are methods for controlling CRW that comprise applying any of the aforementioned compositions provided herein to a plant or a plant part in an amount that provides for inhibition of CRW damage in the plant, plant part, or a plant obtained therefrom relative to infection of a control plant, plant part, or plant obtained therefrom that had not received an application of the composition. In certain embodiments, application of the composition provides for at least about 5%, at least about 10%, at least about 20%, at least about 30%, at least about 40%, at least about 50%, at least about 75%, at least about 85%, or at least about 95% reduction of CRW damage in the plant, plant part, or a plant derived therefrom relative to infection of the control plant, plant part, or plant obtained therefrom. In certain embodiments, the plant part is selected from the group consisting of a leaf, a stem, a flower, a root, a tuber, a pollen grain, and a seed. In certain embodiments, the method further comprises the step of harvesting at least one plant part selected from the group consisting of a leaf, a stem, a flower, a root, a tuber, a pollen grain, or a seed from the plant or plant part. In certain embodiments of any of the aforementioned methods, the methods further comprise obtaining a processed food or feed composition from the plant or plant part. In certain embodiments, the processed food or feed composition is a meal or a paste. In certain embodiments of any of the aforementioned methods, the CRW-active Methylobacterium is selected from the group consisting of ISO13, ISO14, ISO08, NLSO210 and NLS0617. In certain embodiments, the CRW-active Methylobacterium is selected from the group consisting of ISO13, ISO14, ISO08, NLSO210 and NLS0617.

Also provided are methods of making the compositions useful for controlling CRW that comprise combining a CRW-active Methylobacterium with an agriculturally acceptable excipient and/or with an agriculturally acceptable adjuvant. In certain embodiments of the methods, the Methylobacterium sp., is selected from the group consisting of M aminovorans, M. extorquens, M. fujisawaense, M. mesophilicum, M radiotolerans, M rhodesianum, M. nodulans, M phyllosphaerae, M thiocyanatum, and M oryzae. In certain embodiments of the methods, the Methylobacterium is not M. radiotolerans or M. oryzae. In certain embodiments of the methods, the Methylobacterium is adhered to a solid substance. In certain embodiments of the methods, the Methylobacterium is adhered to the solid substance is combined with a liquid to form a composition that is a colloid. In certain embodiments of the methods, the colloid is a gel. In certain embodiments of the methods, the Methylobacterium adhered to the solid substance is provided by culturing the Methylobacterium in the presence of the solid substance. In certain embodiments of the methods, the composition comprises an emulsion. In certain embodiments of the methods, the Methylobacterium is provided by culturing the Methylobacterium in an emulsion. In certain embodiments of any of the aforementioned methods, the CRW-active Methylobacterium is selected from the group consisting of ISO13, ISO14, ISO08, NLSO210 and NLS0617. In certain embodiments, the CRW-active Methylobacterium is selected from the group consisting of ISO13, ISO14, ISO08, NLSO210 and NLS0617.

Methods where Methylobacterium are cultured in biphasic media comprising a liquid phase and a solid substance have been found to significantly increase the resultant yield of Methylobacterium relative to methods where the Methylobacterium are cultured in liquid media alone. In certain embodiments, the methods can comprise growing the Methylobacterium in liquid media with a particulate solid substance that can be suspended in the liquid by agitation under conditions that provide for Methylobacterium growth. In certain embodiments where particulate solid substances are used, at least substantially all of the solid phase can thus be suspended in the liquid phase upon agitation. Such particulate solid substances can comprise materials that are about 1 millimeter or less in length or diameter. In certain embodiments, the degree of agitation is sufficient to provide for uniform distribution of the particulate solid substance in the liquid phase and/or optimal levels of culture aeration. However, in other embodiments provided herein, at least substantially all of the solid phase is not suspended in the liquid phase, or portions of the solid phase are suspended in the liquid phase and portions of the solid phase are not suspended in the liquid phase. Non-particulate solid substances can be used in certain biphasic media where the solid phase is not suspended in the liquid phase. Such non-particulate solid substances include, but are not limited to, materials that are greater than about 1 millimeter in length or diameter. Such particulate and non-particulate solid substances also include, but are not limited to, materials that are porous, fibrous, or otherwise configured to provide for increased surface areas for adherent growth of the Methylobacterium. Biphasic media where portions of the solid phase are suspended in the liquid phase and portions of the solid phase are not suspended in the liquid phase can comprise a mixture of particulate and non-particulate solid substances. Such particulate and non-particulate solid substances used in any of the aforementioned biphasic media also include, but are not limited to, materials that are porous, fibrous, or otherwise configured to provide for increased surface areas for adherent growth of the Methylobacterium. In certain embodiments, the media comprises a colloid formed by a solid and a liquid phase. A colloid comprising a solid and a liquid can be pre-formed and added to liquid media or can be formed in media containing a solid and a liquid. Colloids comprising a solid and a liquid can be formed by subjecting certain solid substances to a chemical and/or thermal change. In certain embodiments, the colloid is a gel. In certain embodiments, the liquid phase of the media is an emulsion. In certain embodiments, the emulsion comprises an aqueous liquid and a liquid that is not miscible, or only partially miscible, in the aqueous liquid. Liquids that are not miscible, or only partially miscible, in water include, but are not limited to, any of the following: (1) liquids having a miscibility in water that is equal to or less than that of pentanol, hexanol, or heptanol at 25 degrees C.; (2) liquids comprising an alcohol, an aldehyde, a ketone, a fatty acid, a phospholipid, or any combination thereof; (3) alcohols selected from the group consisting of aliphatic alcohols containing at least 5 carbons and sterols; (4) an animal oil, microbial oil, synthetic oil, plant oil, or combination thereof, and/or, (5) a plant oil is selected from the group consisting of corn, soybean, cotton, peanut, sunflower, olive, flax, coconut, palm, rapeseed, sesame seed, safflower, and combinations thereof. In certain embodiments, the immiscible or partially immiscible liquid can comprise at least about 0.02% to about 20% of the liquid phase by mass. In certain embodiments, the methods can comprise obtaining a biphasic culture media comprising the liquid, the solid, and Methylobacterium and incubating the culture under conditions that provide for growth of the Methylobacterium. Biphasic culture medias comprising the liquid, the solid, and Methylobacterium can be obtained by a variety of methods that include, but are not limited to, any of. (a) inoculating a biphasic media comprising the liquid and the solid substance withMethylobacterium; (b) inoculating the solid substance with Methylobacterium and then introducing the solid substance comprising the Methylobacterium into the liquid media; (c) inoculating the solid substance with Methylobacterium, incubating the Methylobacterium on the solid substance, and then introducing the solid substance comprising the Methylobacterium into the liquid media; or (d) any combination of (a), (b), or (c). Methods and compositions for growing Methylobacterium in biphasic media comprising a liquid and a solid are disclosed in co-assigned US Patent Application Publication No. 20130324407, which is incorporated herein by reference in its entirety. Compositions comprising dried formulations of Methylobacterium that are adhered to solid substances, methods for making such compositions, and methods of applying those compositions to plants and plant parts including seeds are disclosed in co-assigned U.S. Provisional Patent Application No. 62/051,028, filed Sep. 16, 2014, which is incorporated herein by reference in its entirety.

Methods where Methylobacterium are cultured in media comprising an emulsion have also been found to significantly increase the resultant yield of Methylobacterium relative to methods where the Methylobacterium are cultured in liquid media alone. In certain embodiments, the methods for making the compositions provided herein can comprise growing the CRW-active Methylobacterium agent in an emulsion under conditions that provide for Methylobacterium growth. Medias comprising the emulsion and CRW-active Methylobacterium can be obtained by a variety of methods that include, but are not limited to, any of: (a) inoculating a media comprising the emulsion with Methylobacterium; (b) inoculating the aqueous liquid with the Methylobacterium, introducing the non-aqueous liquid, and mixing to form an emulsion; (c) inoculating the aqueous liquid with the Methylobacterium, introducing the non-aqueous liquid, and mixing to form an emulsion; or (d) any combination of (a), (b), or (c). In certain embodiments, the emulsion comprises an aqueous liquid and a liquid that is not miscible, or only partially miscible, in the aqueous liquid. Non-aqueous liquids that are not miscible, or only partially miscible, in water include, but are not limited to, any of the following: (1) liquids having a miscibility in water that is equal to or less than that of n-pentanol, n-hexanol, or n-heptanol at 25 degrees C.; (2) liquids comprising an alcohol, an aldehyde, a ketone, a fatty acid, a phospholipid, or any combination thereof, (3) alcohols is selected from the group consisting of aliphatic alcohols containing at least 5, 6, or 7 carbons and sterols; (4) an animal oil, microbial oil, synthetic oil, plant oil, or combination thereof, and/or, (5) a plant oil is selected from the group consisting of corn, soybean, cotton, peanut, sunflower, olive, flax, coconut, palm, rapeseed, sesame seed, safflower, and combinations thereof. In certain embodiments, the immiscible or partially immiscible non-aqueous liquid can comprise at least about 0.02% to about 20% of the emulsion by mass. In certain embodiments, the immiscible or partially immiscible non-aqueous liquid can comprise at least about any of about 0.05%, 0.1%, 0.5%, or 1% to about 3%, 5%, 10%, or 20% of the emulsion by mass. Methods and compositions for growing Methylobacterium in media comprising an emulsion are disclosed in co-assigned International Patent Application PCT/US14/40218, filed May 30, 2014, which is incorporated herein by reference in its entirety.

In certain embodiments, the fermentation broth, fermentation broth product, or compositions that comprise CRW-active Methylobacterium sp. can further comprise one or more introduced microorganisms of pre-determined identity other than Methylobacterium. Other microorganisms that can be added include, but are not limited to, microorganisms that are biopesticidal or provide some other benefit when applied to a plant or plant part. Biopesticidal or otherwise beneficial microorganisms thus include, but are not limited to, various Bacillus sp., Pseudomonas sp., Coniothyrium sp., Pantoea sp., Streptomyces sp., and Trichoderma sp. Microbial biopesticides can be a bacterium, fungus, virus, or protozoan. Particularly useful biopesticidal microorganisms include various Bacillus subtilis, Bacillus thuringiensis, Bacillus pumilis, Pseudomonas syringae, Trichoderma harzianum, Trichoderma virens, and Streptomyces lydicus strains. Other microorganisms that are added can be genetically engineered or other isolates that are available as pure cultures. In certain embodiments, it is anticipated that the bacterial or fungal microorganism can be provided in the fermentation broth, fermentation broth product, or composition in the form of a spore.

In certain embodiments, the CRW-active Methylobacterium sp. and compositions comprising the same that are provided herein can be used in conjunction with transgenic plants that express gene products that are inhibitory to growth of certain CRW. Such transgenic plants include, but are not limited to, those expressing interfering RNA molecules (U.S. Patent Appl. No. 20130291188; PCT Appl. No. WO2007/035650), coleopteran active Cry3Bb1 proteins (U.S. Patent Appl. No. 20130031679; U.S. Pat. No. 7,227,056), modified Cry3A proteins (U.S. Patent Appl. Nos. 20120185973, 20130291188, and 20130116170), cry8-like proteins (U.S. Patent Appl. No 20090291896), DIG-10 toxins (U.S. Patent Appl. No. 20130247254), insecticidal secreted proteins (ISPs: U.S. Pat. No. 7,091,399); and one or both of the Cry34Ab1 and Cry35Ab1 proteins and variants thereof (U.S. Pat. Nos. 6,127,180, 6,624,145, 6,340,593, 6,083,499, 6,548,291 and 6,340,593; U.S. Pat. Appl. Nos. 20110275557, 20110154526, 20110154525). Each of the aforementioned patents and patent applications cited in reference to such transgenic plants is incorporated herein by reference in their entireties.

In certain embodiments, the CRW-active Methylobacterium sp. and compositions comprising the same that are provided herein can be used in conjunction with, or comprise, insecticides that also provide for inhibition of CRW growth and/or reductions in CRW-mediated plant damage. Such insecticides can be used in soil treatments (drenches, in furrow deposits, and the like) and/or in seed treatments. In certain embodiments, the insecticide is selected from the group consisting of pyrethrins, synthetic pyrethroids, oxadiazines, chloronicotinyls, neonicotinoids, nitroguanidine insecticides, triazoles, organophosphates, pyrrols, pyrazoles, diacylhydrazines, biological/fermentation products, and carbamates. In certain embodiments, the seed is treated with one or more of the aforementioned insecticides (U.S. Pat. Nos. 6,660,690, and 8,080,496, each incorporated herein by reference in their entireties). Commercial soil applied insecticide formulations that can be used in conjunction with the CRW-active Methylobacterium sp. provided herein include, but are not limited to, various FORCE™ (Amvac Chemical Corp, CA, USA), AZTEC™ (Amvac Chemical Corp, CA, USA), COUNTER™ (Amvac Chemical Corp, CA, USA), FORTRESS™ (Amvac Chemical Corp, CA, USA), FURADAN™ (FMC Corporation, PA, USA), GAUCHO™ (Bayer CropScience, NC, USA), PONCHO™ (Bayer CropScience, NC, USA), LORSBAN™ (Dow Agrosciences, IN, USA), REGENT™ (BASF Corporation, NC, USA), and THIMET™ (Amvac Chemical Corp, CA, USA) formulations. Combinations of the aforementioned insecticides and the aforementioned transgenic plants that provide for inhibition of CRW growth and/or reductions in CRW-mediated plant damage can also be used in conjunction with the CRW-active Methylobacterium sp. provided herein.

In certain embodiments, the liquid culture medium is prepared from inexpensive and readily available components, including, but not limited to, inorganic salts such as potassium phosphate, magnesium sulfate and the like, carbon sources such as glycerol, methanol, glutamic acid, aspartic acid, succinic acid and the like, and amino acid blends such as peptone, tryptone, and the like. Non-limiting examples of liquid media that can be used include, but are not limited to, ammonium mineral salts (AMS) medium (Whittenbury et al., 1970), Vogel-Bonner (VB) minimal culture medium (Vogel and Bonner, 1956), and LB broth (“Luria-Bertani Broth”).

In general, the solid substance used in the methods and compositions that provide for the efficient growth of Methylobacterium can be any suitable solid substance which is insoluble or only partially soluble in water or aqueous solutions. Such suitable solid substances are also non-bacteriocidal or non-bacteriostatic with respect to CRW-active Methylobacterium sp. when the solid substances are provided in the liquid culture media. In certain embodiments, such suitable solid substances are also solid substances that are readily obtained in sterile form or rendered sterile. Solid substances used herein can be sterilized by any method that provides for removal of contaminating microorganisms and thus include, but are not limited to, methods such as autoclaving, irradiation, chemical treatment, and any combination thereof. These solid substances include substances of animal, plant, microbial, fungal, or mineral origin, manmade substances, or combinations of substances of animal, plant, microbial, fungal, or mineral origin and manmade substances. In certain embodiments, the solid substances are inanimate solid substances. Inanimate solid substances of animal, plant, microbial, or fungal origin can be obtained from animals, plants, microbes, or fungi that are inviable (i.e. no longer living) or that have been rendered inviable. Diatom shells are thus inanimate solid substances when previously associated diatom algae have been removed or otherwise rendered inviable. Since diatom shells are inanimate solid substances, they are not considered to be photosynthetic organisms or photosynthetic microorganisms. In certain embodiments, solid substances include, but are not limited to, sand, silt, soil, clay, ash, charcoal, diatomaceous earth and other similar minerals, ground glass or glass beads, ground ceramic materials, ceramic beads, bentonite, kaolin, talc, perlite, mica, vermiculite, silicas, quartz powder, montmorillonite, and combinations thereof. In certain embodiments, the solid substance can be a polymer or polymeric beads. Polymers that can be used as a solid substance include, but are not limited to, various polysaccharides such as cellulosic polymers and chitinous polymers which are insoluble or only partially soluble in water or aqueous solutions, agar (i.e. galactans), and combinations thereof. In certain embodiments, the solid substance can be an insoluble or only partially soluble salt crystal. Salt crystals that can be used include, but are not limited to, insoluble or only partially soluble carbonates, chromates, sulfites, phosphates, hydroxides, oxides, and sulfides. In certain embodiments, the solid substance can be a microbial cell, fungal cell, microbial spore, or fungal spore. In certain embodiments, the solid substance can be a microbial cell or microbial spore wherein the microbial cell or microbial spore is not a photosynthetic microorganism. In certain embodiments, the microbial cell or microbial spore is not a photosynthetic microorganism, where the photosynthetic microorganism is selected from the group consisting of algae, cyanobacteria, diatoms, Botryococcus braunii, Chlorella, Dunaliella tertiolecta, Gracilaria, Pleurochrysis carterae, Sargassum, and Ulva. In still other embodiments, the solid substance can be an inactivated (i.e. inviable) microbial cell, fungal cell, microbial spore, or fungal spore. In still other embodiments, the solid substance can be a quiescent (i.e. viable but not actively dividing) microbial cell, fungal cell, microbial spore, or fungal spore. In still other embodiments, the solid substance can be cellular debris of microbial origin. In still other embodiments, the solid substance can be particulate matter from any part of a plant. Plant parts that can be used to obtain the solid substance include, but are not limited to, cobs, husks, hulls, leaves, roots, flowers, stems, barks, seeds, and combinations thereof. Products obtained from processed plant parts including, but not limited to, bagasse, wheat bran, soy grits, crushed seed cake, stover, and the like can also be used. Such plant parts, processed plants, and/or processed plant parts can be milled to obtain the solid material in a particulate form that can be used. In certain embodiments, wood or a wood product including, but not limited to, wood pulp, sawdust, shavings, and the like can be used. In certain embodiments, the solid substance can be a particulate matter from an animal(s), including, but not limited to, bone meal, gelatin, ground or powdered shells, hair, macerated hide, and the like.

In certain embodiments, the solid substance is provided in a particulate form that provides for distribution of the solid substance in the culture media. In certain embodiments, the solid substance is comprised of particle of about 2 microns to about 1000 microns in average length or average diameter. In certain embodiments, the solid substance is comprised of particle of about 1 microns to about 1000 microns in average length or average diameter. In certain embodiments, the solid substance is a particle of about 1, 2, 4, 10, 20, or 40 microns to any of about 100, 200, 500, 750, or 1000 microns in average length or average diameter. Desirable characteristics of particles used in the methods and compositions provided herein include suitable wettability such that the particles can be suspended throughout the media upon agitation.

In certain embodiments, the solid substance is provided in the media as a colloid wherein the continuous phase is a liquid and the dispersed phase is the solid. Suitable solids that can be used to form colloids in liquid media used to grow CRW-active Methylobacterium sp. include, but are not limited to, various solids that are referred to as hydrocolloids. Such hydrocolloids used in the media, methods and compositions provided herein can be hydrophilic polymers, of plant, animal, microbial, or synthetic origin. Hydrocolloid polymers used in the methods can contain many hydroxyl groups and/or can be polyelectrolytes. Hydrocolloid polymers used in the compositions and methods provided herein include, but are not limited to, agar, alginate, arabinoxylan, carrageenan, carboxymethylcellulose, cellulose, curdlan, gelatin, gellan, β-glucan, guar gum, gum arabic, locust bean gum, pectin, starch, xanthan gum, and mixtures thereof. In certain embodiments, the colloid used in the media, methods, and compositions provided herein can comprise a hydrocolloid polymer and one or more proteins.

In certain embodiments, the solid substance can be a solid substance that provides for adherent growth of the CRW-active Methylobacterium sp. on the solid substance. CRW-active Methylobacterium sp. that are adhered to a solid substance are Methylobacterium that cannot be substantially removed by simply washing the solid substance with the adherent CRW-active Methylobacterium sp. with growth media whereas non-adherent Methylobacterium can be substantially removed by washing the solid substance with liquid growth media. In this context, “substantially removed” means that at least about 30%, 40%, 50%, 60%, 70%, or 80% the Methylobacterium present are removed when the solid substance is washed with three volumes of liquid growth media. Such washing can be effected by a variety of methods including, but not limited to, decanting liquid from a washed solid phase or passing liquid through a solid phase on a filter that permits flow through of bacteria in the liquid. In certain embodiments, the adherent CRW-active Methylobacterium sp. that are associated with the solid can include both Methylobacterium that are directly attached to the solid and/or Methylobacterium that are indirectly attached to the solid substance. Methylobacterium that are indirectly attached to the solid substance include, but are not limited to, Methylobacterium that are attached to another Methylobacterium or to another microorganism that is attached to the solid substance, Methylobacterium that are attached to the solid substance by being attached to another substance that is attached to the solid substance, and the like. In certain embodiments, at least 10%, 20%, 30%, 40%, 50%, 60%, 70%, 80%, 90%, 95%, 98%, 99%, 99.5% or 99.9% of the Methylobacterium in the fermentation broth, fermentation broth product, or compositions are Methylobacterium that are adhered to the solid substance. In certain embodiments, adherent CRW-active Methylobacterium sp. can be present on the surface of the solid substance in the fermentation broth, fermentation broth product, or composition at a density of at least about 1 Methylobacterium/20 square micrometers, of at least about 1 Methylobacterium/10 square micrometers, of at least about 1 Methylobacterium/10 square micrometers, of at least about 1 Methylobacterium/5 square micrometers, of at least about 1 Methylobacterium/2 square micrometers, or of at least about 1 Methylobacterium/square micrometer. In certain embodiments, adherent CRW-active Methylobacterium sp. can be present on the surface of the solid substance in the fermentation broth, fermentation broth product, or composition at a density of at least about 1 Methylobacterium/20 square micrometers to about 1 Methylobacterium/square micrometer, of at least about 1 Methylobacterium/10 square micrometers to about 1 Methylobacterium/square micrometer, of at least about 1 Methylobacterium/10 square micrometers to about 1 Methylobacterium/square micrometer, of at least about 1 Methylobacterium/5 square micrometers to about 1 Methylobacterium/square micrometer, or of at least about 1 Methylobacterium/2 square micrometers to about 1 Methylobacterium/square micrometer. In certain embodiments, adherent CRW-active Methylobacterium sp. can be present on the surface of the solid substance in the fermentation broth, fermentation broth product, or composition at a density of at least about 1 Methylobacterium/20 square micrometers to about 1 Methylobacterium/2 square micrometers, of at least about 1 Methylobacterium/10 square micrometers to about 1 Methylobacterium/2 square micrometers, of at least about 1 Methylobacterium/10 square micrometers to about 1 Methylobacterium/2 square micrometers, or of at least about 1 Methylobacterium/5 square micrometers to about 1 Methylobacterium/2 square micrometers. Biphasic fermentation broths provided herein can comprise a liquid phase that contains non-adherent Methylobacterium. In certain embodiments, titers of non-adherent Methylobacterium in the liquid phase can be less than about 100,000, 10,000, or 1,000 CFU/ml.

In certain embodiments, the CRW-active Methylobacterium is selected from the group consisting of IS013, IS014, IS008, NLSO210 and NLS0617. In certain embodiments, the CRW-active Methylobacterium is selected from the group consisting of IS013, IS014, IS008, NLSO210 and NLS0617.

Biphasic culture methods provided can yield fermentation broths with CRW-active Methylobacterium sp. at a titer of greater than about 5×10⁸ colony-forming units per milliliter, at a titer of greater than about 1×10⁹ colony-forming units per milliliter, at a titer of greater than about 1×10¹⁰ colony-forming units per milliliter, at a titer of at least about 3×10¹⁰ colony-forming units per milliliter. In certain embodiments, fermentation broths provided herein can comprise CRW-active Methylobacterium sp. at a titer of at least about 5×10⁸ colony-forming units per milliliter to at least about 3×10¹⁰ colony-forming units per milliliter, at least about 5×10⁸ colony-forming units per milliliter to at least about 4×10¹⁰ colony-forming units per milliliter, or at least about 5×10⁸ colony-forming units per milliliter to at least about 6×10¹⁰ colony-forming units per milliliter. In certain embodiments, fermentation broths provided herein can comprise CRW-active Methylobacterium sp. at a titer of at least about 1×10⁹ colony-forming units per milliliter to at least about 3×10¹⁰ colony-forming units per milliliter, at least about 1×10⁹ colony-forming units per milliliter to at least about 4×10¹⁰ colony-forming units per milliliter, or at least about 1×10⁹ colony-forming units per milliliter to at least about 6×10¹⁰ colony-forming units per milliliter. In certain embodiments, fermentation broths provided herein will comprise CRW-active Methylobacterium sp. at a titer of at least about 1×10¹⁰ colony-forming units per milliliter to at least about 3×10¹⁰ colony-forming units per milliliter, at least about 1×10¹⁰ colony-forming units per milliliter to at least about 4×10¹⁰ colony-forming units per milliliter, or at least about 1×10¹⁰ colony-forming units per milliliter to at least about 6×10¹⁰ colony-forming units per milliliter. In certain embodiments, fermentation broths provided herein will comprise CRW-active Methylobacterium sp. at a titer of, at least about 3×10¹⁰ colony-forming units per milliliter to at least about 4×10¹⁰ colony-forming units per milliliter, or at least about 3×10¹⁰ colony-forming units per milliliter to at least about 6×10¹⁰ colony-forming units per milliliter. In certain embodiments, the CRW-active Methylobacterium is selected from the group consisting of ISO13, ISO14, ISO08, NLSO210 and NLS0617. In certain embodiments, the CRW-active Methylobacterium is selected from the group consisting of ISO13, ISO14, ISO08, NLSO210 and NLS0617.

Solid substances with adherent CRW-active Methylobacterium sp. can be obtained as fermentation products can be used to make various compositions useful for treating plants or plant parts to inhibit CRW growth or reduce CRW damage to a plant. Alternatively, compositions provided herein comprising CRW-active Methylobacterium sp., solid substances with CRW-active Methylobacterium sp. grown thereon, or comprising emulsions with CRW-active Methylobacterium sp. grown therein can be used to treat plants or plant parts. Plants, plant parts, and, in particular, plant seeds that have been at least partially coated or coated with the fermentation broth products or compositions comprising CRW-active Methylobacterium sp. are thus provided. Also provided are processed plant products that contain the fermentation broth products or compositions with CRW-active Methylobacterium sp. or adherent CRW-active Methylobacterium sp. Solid substances with adherent CRW-active Methylobacterium sp. can be used to make various compositions that are particularly useful for treating plant seeds. Seeds that have been at least partially coated with the fermentation broth products or compositions are thus provided. Also provided are processed seed products, including, but not limited to, meal, flour, feed, and flakes that contain the fermentation broth products or compositions provided herein. In certain embodiments, the processed plant product will be non-regenerable (i.e. will be incapable of developing into a plant). In certain embodiments, the solid substance used in the fermentation product or composition that at least partially coats the plant, plant part, or plant seed or that is contained in the processed plant, plant part, or seed product comprises a solid substance and associated or adherent CRW-active Methylobacterium sp. that can be readily identified by comparing a treated and an untreated plant, plant part, plant seed, or processed product thereof. In certain embodiments, the CRW-active Methylobacterium is selected from the group consisting of ISO13, ISO14, ISO08, NLSO210 and NLS0617. In certain embodiments, the CRW-active Methylobacterium is selected from the group consisting of ISO13, ISO14, ISO08, NLSO210 and NLS0617.

Compositions useful for treating plants or plant parts that comprise CRW-active Methylobacterium sp., a solid substance with adherent CRW-active Methylobacterium sp., or comprising emulsions with CRW-active Methylobacterium sp. grown therein can also further comprise an agriculturally acceptable adjuvant or an agriculturally acceptable excipient. An agriculturally acceptable adjuvant or an agriculturally acceptable excipient is typically an ingredient that does not cause undue phytotoxicity or other adverse effects when exposed to a plant or plant part. In certain embodiments, the solid substance can itself be an agriculturally acceptable adjuvant or an agriculturally acceptable excipient so long as it is not bacteriocidal or bacteriostatic to the Methylobacterium. In other embodiments, the composition further comprises at least one of an agriculturally acceptable adjuvant or an agriculturally acceptable excipient. Any of the aforementioned compositions can also further comprise a pesticide. Pesticides used in the composition include, but are not limited to, an insecticide, a fungicide, a nematocide, and a bacteriocide. In certain embodiments, the pesticide used in the composition is a pesticide that does not substantially inhibit growth of the Methylobacterium. As Methylobacterium are gram negative bacteria, suitable bacteriocides used in the compositions can include, but are not limited to, bacteriocides that exhibit activity against gram positive bacteria but not gram negative bacteria. Compositions provided herein can also comprise a bacteriostatic agent that does not substantially inhibit growth of the Methylobacterium. Bacteriostatic agents suitable for use in compositions provided herein include, but are not limited to, those that exhibit activity against gram positive bacteria but not gram negative bacteria. Any of the aforementioned compositions can also be an essentially dry product (i.e. having about 5% or less water content), a mixture of the composition with an emulsion, or a suspension.

Agriculturally acceptable adjuvants used in the compositions that comprise CRW-active Methylobacterium sp. include, but are not limited to, components that enhance product efficacy and/or products that enhance ease of product application. Adjuvants that enhance product efficacy can include various wetters/spreaders that promote adhesion to and spreading of the composition on plant parts, stickers that promote adhesion to the plant part, penetrants that can promote contact of the active agent with interior tissues, extenders that increase the half-life of the active agent by inhibiting environmental degradation, and humectants that increase the density or drying time of sprayed compositions. Wetters/spreaders used in the compositions can include, but are not limited to, non-ionic surfactants, anionic surfactants, cationic surfactants, amphoteric surfactants, organo-silicate surfactants, and/or acidified surfactants. Stickers used in the compositions can include, but are not limited to, latex-based substances, terpene/pinolene, and pyrrolidone-based substances. Penetrants can include mineral oil, vegetable oil, esterified vegetable oil, organo-silicate surfactants, and acidified surfactants. Extenders used in the compositions can include, but are not limited to, ammonium sulphate, or menthene-based substances. Humectants used in the compositions can include, but are not limited to, glycerol, propylene glycol, and diethyl glycol. Adjuvants that improve ease of product application include, but are not limited to, acidifying/buffering agents, anti-foaming/de-foaming agents, compatibility agents, drift-reducing agents, dyes, and water conditioners. Anti-foaming/de-foaming agents used in the compositions can include, but are not limited to, dimethopolysiloxane. Compatibility agents used in the compositions can include, but are not limited to, ammonium sulphate. Drift-reducing agents used in the compositions can include, but are not limited to, polyacrylamides, and polysaccharides. Water conditioners used in the compositions can include, but are not limited to, ammonium sulphate.

Methods of treating plants and/or plant parts with the fermentation broths, fermentation broth products, and compositions comprising CRW-active Methylobacterium sp. are also provided herein. Treated plants, and treated plant parts obtained therefrom, include, but are not limited to, corn, Brassica sp. (e.g., B. napus, B. rapa, B. juncea), alfalfa, rice, rye, sorghum, millet (e.g., pearl millet (Pennisetum glaucum), proso millet (Panicum miliaceum), foxtail millet (Setaria italica), finger millet (Eleusine coracana), sunflower, safflower, soybean, tobacco, potato, peanuts, cotton, sweet potato (Ipomoea batatus), cassava, coffee, coconut, pineapple, citrus trees, cocoa, tea, banana, avocado, fig, guava, mango, olive, papaya, cashew, macadamia, almond, sugar beets, sugarcane, oats, barley, tomatoes lettuce, green beans, lima beans, peas, cucurbits such as cucumber, cantaloupe, and musk melon, ornamentals, and conifers. Plant parts that are treated include, but are not limited to, leaves, stems, flowers, roots, seeds, fruit, tubers, coleoptiles, and the like. Ornamental plants and plant parts that can be treated include, but are not limited to azalea, hydrangea, hibiscus, roses, tulips, daffodils, petunias, carnation, poinsettia, and chrysanthemum. Seeds or other propagules of any of the aforementioned plants can be treated with the fermentation broths, fermentation broth products, fermentation products, and/or compositions provided herein.

In certain embodiments, plants and/or plant parts are treated by applying the fermentation broths, fermentation broth products, fermentation products, and compositions that comprise CRW-active Methylobacterium sp. as a spray. Such spray applications include, but are not limited to, treatments of a single plant part or any combination of plant parts. Spraying can be achieved with any device that will distribute the fermentation broths, fermentation broth products, fermentation products, and compositions to the plant and/or plant part(s). Useful spray devices include a boom sprayer, a hand or backpack sprayer, crop dusters (i.e. aerial spraying), and the like. Spraying devices and or methods providing for application of the fermentation broths, fermentation broth products, fermentation products, and compositions to either one or both of the adaxial surface and/or abaxial surface can also be used. Plants and/or plant parts that are at least partially coated with any of a biphasic fermentation broth, a fermentation broth product, fermentation product, or compositions that comprise a solid substance with CRW-active Methylobacterium sp. adhered thereto are also provided herein. Also provided herein are processed plant products that comprise a solid substance with CRW-active Methylobacterium sp. adhered thereto.

In certain embodiments, seeds are treated by exposing the seeds to the fermentation broths, fermentation broth products, fermentation products, and compositions that comprise CRW-active Methylobacterium sp. Seeds can be treated with the fermentation broths, fermentation broth products, and compositions provided herein by methods including, but not limited to, imbibition, coating, spraying, and the like. Seed treatments can be effected with both continuous and/or a batch seed treaters. In certain embodiments, the coated seeds can be prepared by slurrying seeds with a coating composition containing a fermentation broth, fermentation broth product, or compositions that comprise the solid substance with CRW-active Methylobacterium sp. and air drying the resulting product. Air drying can be accomplished at any temperature that is not deleterious to the seed or the Methylobacterium, but will typically not be greater than 30 degrees Centigrade. The proportion of coating that comprises a solid substance and CRW-active Methylobacterium sp. includes, but is not limited to, a range of 0.1 to 25% by weight of the seed, 0.5 to 5% by weight of the seed, and 0.5 to 2.5% by weight of seed. In certain embodiments, a solid substance used in the seed coating or treatment will have CRW-active Methylobacterium sp. adhered thereon. In certain embodiments, a solid substance used in the seed coating or treatment will be associated with CRW-active Methylobacterium sp. and will be a fermentation broth, fermentation broth product, or composition obtained by the methods provided herein. Various seed treatment compositions and methods for seed treatment disclosed in U.S. Pat. Nos. 5,106,648, 5,512,069, and 8,181,388 are incorporated herein by reference in their entireties and can be adapted for use with an active agent comprising the fermentation broths, fermentation broth products, or compositions provided herein. In certain embodiments, the composition used to treat the seed can contain agriculturally acceptable excipients that include, but are not limited to, woodflours, clays, activated carbon, diatomaceous earth, fine-grain inorganic solids, calcium carbonate and the like. Clays and inorganic solids that can be used with the fermentation broths, fermentation broth products, or compositions provided herein include, but are not limited to, calcium bentonite, kaolin, china clay, talc, perlite, mica, vermiculite, silicas, quartz powder, montmorillonite and mixtures thereof. Agriculturally acceptable adjuvants that promote sticking to the seed that can be used include, but are not limited to, polyvinyl acetates, polyvinyl acetate copolymers, hydrolyzed polyvinyl acetates, polyvinylpyrrolidone-vinyl acetate copolymer, polyvinyl alcohols, polyvinyl alcohol copolymers, polyvinyl methyl ether, polyvinyl methyl ether-maleic anhydride copolymer, waxes, latex polymers, celluloses including ethylcelluloses and methylcelluloses, hydroxy methylcelluloses, hydroxypropylcellulose, hydroxymethylpropylcelluloses, polyvinyl pyrrolidones, alginates, dextrins, malto-dextrins, polysaccharides, fats, oils, proteins, karaya gum, jaguar gum, tragacanth gum, polysaccharide gums, mucilage, gum arabics, shellacs, vinylidene chloride polymers and copolymers, soybean-based protein polymers and copolymers, lignosulfonates, acrylic copolymers, starches, polyvinylacrylates, zeins, gelatin, carboxymethylcellulose, chitosan, polyethylene oxide, acrylimide polymers and copolymers, polyhydroxyethyl acrylate, methylacrylimide monomers, alginate, ethylcellulose, polychloroprene and syrups or mixtures thereof. Other useful agriculturally acceptable adjuvants that can promote coating include, but are not limited to, polymers and copolymers of vinyl acetate, polyvinylpyrrolidone-vinyl acetate copolymer and water-soluble waxes. Various surfactants, dispersants, anticaking-agents, foam-control agents, and dyes disclosed herein and in U.S. Pat. No. 8,181,388 can be adapted for use with an active agent comprising the fermentation broths, fermentation broth products, or compositions provided herein.

Provided herein are compositions that comprise CRW-active Methylobacterium sp. that provide control of CRW damage to plants, plant parts, and plants obtained therefrom relative to untreated plants, plant parts, and plants obtained therefrom that have not been exposed to the compositions. In certain embodiments, plant parts, including, but not limited to, a seed, a leaf, a fruit, a stem, a root, a tuber, a pollen grain, or a coleoptile can be treated with the compositions provided herein to inhibit of CRW growth and/or reduce of CRW damage to a plant. Treatments or applications can include, but are not limited to, spraying, coating, partially coating, immersing, and/or imbibing the plant or plant parts with the compositions provided herein. In certain embodiments, a seed, a leaf, a fruit, a stem, a root, a tuber, or a coleoptile can be immersed and/or imbibed with a liquid, semi-liquid, emulsion, or slurry of a composition provided herein. Such seed immersion or imbibition can be sufficient to provide for inhibition of CRW growth and/or reductions in CRW damage in a treated plant or plant part in comparison to an untreated plant or plant part. Such for inhibition of CRW growth and/or reductions in CRW damage includes, but is not limited to decreases in larval growth, inhibition of larval development, disruption of larval feeding behaviors, and/or reductions in damage to roots, tubers, or other plant parts relative to untreated plants. In certain embodiments, plant seeds can be immersed and/or imbibed for at least 1, 2, 3, 4, 5, or 6 hours. Such immersion and/or imbibition can, in certain embodiments, be conducted at temperatures that are not deleterious to the plant seed or the Methylobacterium. In certain embodiments, the seeds can be treated at about 15 to about 30 degrees Centigrade or at about 20 to about 25 degrees Centigrade. In certain embodiments, seed imbibition and/or immersion can be performed with gentle agitation.

In certain embodiments where plant seeds are treated with Methylobacterium compositions provided herein, the compositions further comprise one or more lubricants to ensure smooth flow and separation (singulation) of seeds in the seeding mechanism, for example a planter box. Lubricants for use in such compositions include talc, graphite, polyethylene wax based powders (such as Fluency Agent), protein powders, for example soybean protein powders, or a combination of protein powders and a lipid, for example lecithin or a vegetable oil. Lubricants can be applied to seeds simultaneously with application of Methylobacterium, or may be mixed with Methylobacterium prior to application of the compositions to the seeds.

Amounts of the compositions that comprise CRW-active Methylobacterium sp. sufficient to provide for a reduction in CRW damage of a plant or plant part can thus be determined by measuring any or all of changes in CRW feeding behavior, CRW growth and/or the adverse effects of CRW feeding in treated plants or plant parts relative to untreated plants or plant parts. Adverse effects of CRW growth in a plant that can be measured include any type of plant tissue damage or necrosis, any type of plant yield reduction, any reduction in the value of the crop plant product, and/or production of undesirable fungal metabolites or fungal growth by-products including but not limited to mycotoxins. In certain embodiments, an Iowa 1-6 CRW damage rating system where a value of 1 equals no injury or only a few minor feeding scars, a value of 2 equals feeding injury evident, but no roots eaten back to 11/2 inches of the plant, a value of 3 equals at least one root eaten off to within 11/2 inches of the plant, but never an entire node of roots destroyed, a value of 4 equals one node of roots eaten back to within 11/2 inches of the plant, a value of 5 equals two nodes of roots eaten back to within 11/2 inches of the plant, and a value of 6 equals three nodes of roots eaten back to within 11/2 inches of the plant can also be used to determine amounts of the compositions sufficient to provide for a reduction in CRW damage to a plant or plant part. Mycotoxins comprise a number of toxic molecules produced by fungal species, including but not limited to polyketides (including aflatoxins, demethylsterigmatocystin, O-methylsterigmatocystin etc.), fumonisins, alperisins (e.g., A₁, A₂, B₁, B₂), sphingofungins (A, B, C and D), trichothecenes, fumifungins, and the like. Methods of quantitating mycotoxin levels are widely documented. Moreover, commercial kits for measurement of the mycotoxins such as aflatoxin, fumonisin, deoxynivalenol, and zearalenone are also available (VICAM, Watertown, MA, USA).

Compositions provided herein comprising CRW-active Methylobacterium sp. are therefore expected to be useful in inhibiting CRW growth and/or reducing CRW damage in a wide variety of plants, including, but not limited to: corn, cucumber, cantaloupe, squash, gourd, and pumpkin, common bean, lima bean, sweet potato, soybean, and winged bean, tomato, potato, cassava, rice, sorghum, wheat, cabbage, peanut, watermelon, bell pepper, pea, beet, okra, onion, and lettuce. Compositions provided herein comprising CRW-active Methylobacterium sp. are also expected to be useful in inhibiting growth and/or reducing damage caused by Diabrotica balteata, D. virgifera zea Krysan & Smith, Diabrotica barberi, Diabrotica undecimpunctata and/or Diabrotica virgifera species.

In certain embodiments, an amount of a composition provided herein that is sufficient to provide for inhibition of CRW damage in a plant or plant part can be a composition with CRW-active Methylobacterium sp. at a titer of at least about 1×10⁴ colony-forming units per milliliter, at least about 1×10⁵ colony-forming units per milliliter, at least about 1×10⁶ colony-forming units per milliliter, at least about 5×10⁶ colony-forming units per milliliter, at least about 1×10⁷ colony-forming units per milliliter, at least about 5×10⁸ colony-forming units per milliliter, at least about 1×10⁹ colony-forming units per milliliter, at least about 1×10¹⁰ colony-forming units per milliliter, or at least about 3×10¹⁰ colony-forming units per milliliter. In certain embodiments, an amount of a composition provided herein that is sufficient to provide for inhibition of CRW growth and/or reduction of CRW damage to a plant or plant part can be a composition with CRW-active Methylobacterium sp. at a titer of at least about 1×10⁴ colony-forming units per milliliter, at least about 1×10⁵ colony-forming units per milliliter, about least about 1×10⁶ colony-forming units per milliliter, at least about 5×10⁶ colony-forming units per milliliter, at least about 1×10⁷ colony-forming units per milliliter, or at least about 5×10⁸ colony-forming units per milliliter to at least about 6×10¹⁰ colony-forming units per milliliter of a liquid or an emulsion. In certain embodiments, an amount of a composition provided herein that is sufficient to provide for inhibition of CRW growth and/or reduction of CRW damage to a plant or plant part can be a fermentation broth product with a CRW-active Methylobacterium sp. titer of a solid phase of that product is at least about 1×10⁴ colony-forming units per gram, at least about 1×10⁵ colony-forming units per gram, at least about 1×10⁶ colony-forming units per gram, at least about 5×10⁶ colony-forming units per gram, at least about 1×10⁷ colony-forming units per gram, or at least about 5×10⁸ colony-forming units per gram to at least about 6×10¹⁰ colony-forming units of Methylobacterium per gram, at least about 1×10¹¹ colony-forming units of Methylobacterium per gram, at least about 1×10¹² colony-forming units of Methylobacterium per gram, at least about 1×10¹³ colony-forming units of Methylobacterium per gram, or at least about 5×10¹³ colony-forming units of Methylobacterium per gram of the solid phase. In certain embodiments, an amount of a composition provided herein that is sufficient to provide for inhibition of CRW growth and/or reduction of CRW damage to a plant or plant part can be a composition with a Methylobacterium titer of at least about 1×10⁶ colony-forming units per gram, at least about 5×10⁶ colony-forming units per gram, at least about 1×10⁷ colony-forming units per gram, or at least about 5×10⁸ colony-forming units per gram to at least about 6×10¹⁰ colony-forming units of Methylobacterium per gram, at least about 1×10¹¹ colony-forming units of Methylobacterium per gram, at least about 1×10¹² colony-forming units of Methylobacterium per gram, at least about 1×10¹³ colony-forming units of Methylobacterium per gram, or at least about 5×10¹³ colony-forming units of Methylobacterium per gram of particles in the composition containing the particles that comprise a solid substance wherein a mono-culture or co-culture of CRW-active Methylobacterium sp. is adhered thereto. In certain embodiments, an amount of a composition provided herein that is sufficient to provide for inhibition of CRW growth and/or reduction of CRW damage to a plant or plant part can be a composition with a Methylobacterium titer of at least about 1×10⁶ colony-forming units per mL, at least about 5×10⁶ colony-forming units per mL, at least about 1×10⁷ colony-forming units per mL, or at least about 5×10⁸ colony-forming units per mL to at least about 6×10¹⁰ colony-forming units of Methylobacterium per mL in a composition comprising an emulsion wherein a mono-culture or co-culture of a CRW-active Methylobacterium sp. adhered to a solid substance is provided therein or grown therein. In certain embodiments, an amount of a composition provided herein that is sufficient to provide for inhibition of CRW growth and/or reduction of CRW damage to a plant or plant part can be a composition with a Methylobacterium titer of at least about 1×10⁶ colony-forming units per mL, at least about 5×10⁶ colony-forming units per mL, at least about 1×10⁷ colony-forming units per mL, or at least about 5×10⁸ colony-forming units per mL to at least about 6×10¹⁰ colony-forming units of Methylobacterium per mL of in a composition comprising an emulsion wherein a mono-culture or co-culture of a CRW-active Methylobacterium sp. is provided therein or grown therein.

### EXAMPLES

The following examples are included to demonstrate certain embodiments. It will be appreciated by those of skill in the art that the techniques disclosed in the following examples represent techniques determined by the Applicants to function well in the practice of the disclosure. However, those of skill in the art should, in light of the instant disclosure, appreciate that many changes can be made in the specific embodiments that are disclosed, while still obtaining like or similar results, without departing from the scope of the invention.

### Example 1. Reductions in CRW-mediated plant lodging and yield loss by Methylobacterium cultures

A corn trial was established at Cropsey IL for the purpose of evaluating 14 PPFM (pink-pigmented-facultative-methylotrophs of the species Methylobacterium) isolates applied as a foliar spray to corn plants at an early vegetative stage (V1) and also at a reproductive stage (R1).

The trial was located in a geographic area of East-Central Illinois that historically has experienced high levels of western corn rootworm (Diabrotica virgifera virgifera LeConte) infestation and attendant reduced yields and damage to corn crops

### Experimental Design

The field trial was conducted as a split design consisting of four 30-inch rows that were each 20 feet long. The two middle rows were the treatment rows, the two outside rows were used as untreated border rows. There were eight replications of each of the 14 PPFM treatments for application at growth stages V3 and R1. The 14 PPFM treatments plus the control (no PPFM treatment) comprised the whole plot, and the growth stage V3 and R1 comprised the split plot. There was a V3 and R1 check (no PPFM control) included in each of the 8 replications.

### Methods

In preparation for the field trials, the PPFM cultures described in Table 1 were grown in media comprising Ammonium Mineral Salts (AMS), glycerol, peptone, and diatomaceous earth (2 grams/liter), at 30° C. for 6 days essentially as described in co-assigned U.S. Patent Application Publication No. US20130324407 and incorporated herein by reference in its entirety. The cultures comprising PPFM exhibiting adherent growth to the diatomaceous earth were then harvested by centrifugation at 5000 rpm for 15 minutes and then re-suspended in AMS + glycerol+peptone with 20% glycerol as a cryoprotectant at 10× concentration. The fermentation products comprising the diatomaceous earth with the adherent Methylobacterium were aliquoted and frozen at −80 until thawed for use.

A corn hybrid containing transgenic events MON88017×MON89034 (GENUITY VT TRIPLE PRO™; Monsanto, St. Louis, MO., USA) was used for protection against insect pests including Western Corn Rootworm (Diabrotica virgifera LeConte). The MON 89034 transgenic insertion event expresses the Cry1A.105 and Cry2Ab2 Bacillus thuringiensis proteins, which confer resistance to lepidopteran insect pests. The MON88017 transgenic insertion event produces an engineered Bacillus thuringiensis Cry3Bb1 protein, which can confer resistance to certain susceptible Corn Rootworm, and a CP4 EPSPS gene that confers tolerance to glyphosate. The corn seed was also treated with PONCHO™ 500 (Bayer Crop Science, North Carolina, USA), an insecticidal seed treatment containing clothianidin for protection against soil insect pests. A fermentation product comprising adherent PPFM that had grown on the diatomaceous earth was applied to the corn at the V3 and R1 stages at a rate of 15 gal per acre using a backpack chemical sprayer. The PPFM application rates are provided below in Tables 2 and 3.

The trial was managed with local agronomic practices throughout the growing season (glyphosate herbicide was applied at V4 stage and nitrogen (N) fertilizer applied at 140 lbs/acre, etc) and harvested for yield with a commercial harvest combine.

TABLE 2
Titers of PPFMs Applied at the R1 Stage
at Indicated Locations (in CFU/mL)

| | | Cropsey |
|---|---|---|
| NLS # | Isolate | Titer |
| 0046 | ISO01 | 8.6E+08 |
| 0020 | ISO02 | 1.2E+09 |
| 0017 | ISO03 | 2.8E+08 |
| 0042 | ISO04 | 2.4E+08 |
| 0089 | ISO05 | 6.7E+08 |
| 0068 | ISO06 | 3.1E+08 |
| 0065 | ISO07 | 3.8E+08 |
| 0069 | ISO08 | 2.0E+08 |
| 0062 | ISO09 | 1.0E+08 |
| 0064 | ISO10 | 8.9E+08 |
| 0021 | ISO11 | 9.7E+07 |
| 0066 | ISO12 | 5.6E+08 |
| 0037 | ISO13 | ND¹ |
| 0038 | ISO14 | 1.3E+08 |

¹ND: Not determined.

TABLE 3
Titers of PPFMs Applied at the V3 Stage
at Indicated Locations (in CFU/mL)

| | | Cropsey |
|---|---|---|
| NLS # | Isolate | Titer |
| 0046 | ISO01 | 5.3E+08 |
| 0020 | ISO02 | 1.0E+09 |
| 0017 | ISO03 | 4.4E+08 |
| 0042 | ISO04 | 5.6E+08 |
| 0089 | ISO05 | 7.0E+07 |
| 0068 | ISO06 | 2.9E+08 |
| 0065 | ISO07 | 3.7E+08 |
| 0069 | ISO08 | 4.3E+08 |
| 0062 | ISO09 | ND¹ |
| 0064 | ISO10 | 1.1E+09 |
| 0021 | ISO11 | ND¹ |
| 0066 | ISO12 | 2.9E+08 |
| 0037 | ISO13 | 1.5E+08 |
| 0038 | ISO14 | 2.4E+08 |

¹ND: Not determined.

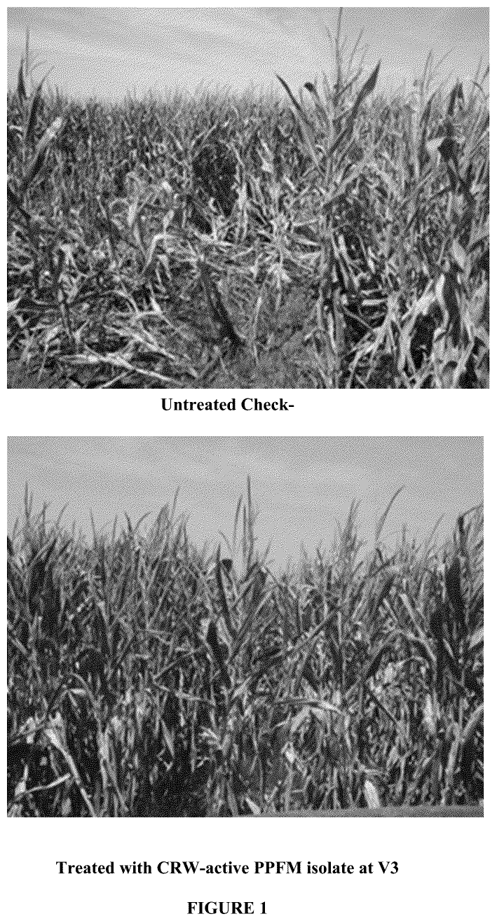

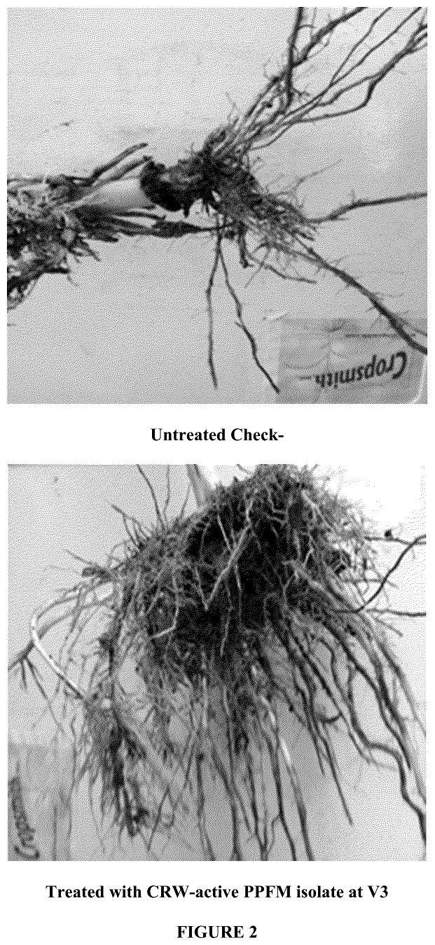

Approximately 14 days after the R1 stage, substantial corn root lodging occurred in certain plants but did not occur in other plants treated with certain PPFM isolates (FIG. 1). Root lodging occurs when the roots cannot keep the plant upright in the face of strong winds, and the plants lean over- or lodge and is a known outcome of corn rootworm feeding on nodal roots. Inspection of the nodal roots of the untreated check plants showed evidence of CRW damage to the nodal roots, indicating that the field test contained CRW that were resistant to the Cry3Bb1 protein (FIG. 2).

A percent lodging rating was taken in the plots to determine if any of the PPFM isolates had an effect on lodging. These ratings were transformed using the arcsine square root transformation (square root of (% lodged/100)), which is a standard transformation for binomial proportions. The plots were harvested for bushel/acre yield with a commercial harvest combine. Trial data were collected, entered into EXCEL™ (Microsoft Corp., Seattle, WA), and analyzed using the ANALYZE/FIT MODEL platform in JMP (JMP software Version 10.0.1 from SAS Institute Inc.) Analyses within each site were conducted using analysis of variance with fixed treatment effects and random effects for replicates. Means of the treatments were compared using pairwise contrasts of the least-squares means from the ANOVA model within JMP.

### Results

For the transformed lodging rating data (Table 4), the analyses showed that, at stage V3, seven of the fourteen isolates had a lower mean transformed value than the control, and the differences were significant at the p=0.2 level, isolate 3 showed the greatest reduction in root lodging (p=0.06). We observed that the control plants had visibly less roots, and evidence of corn rootworm larval feeding. The plants treated with a CRW-active Methylobacterium isolate had a much more vigorous and intact root system. The difference in root masses between these two treatments can be seen in FIG. 2. There were no isolates that showed a reduction in lodging vs the check at the R1 application stage. One isolate, isolate 14, had a significantly higher mean than the check (p=0.017) at the R1 stage.

TABLE 4
Effect of fourteen PPFM isolates on root lodging rating,
arc-sine transformed data

| | | % | | | | |
|---|---|---|---|---|---|---|
| | | Lodging | | Arc | Δ | |
| PPFM | | rating | Std | sine | (Isolate − | P |
| Isolate | Stage | means | Err | means | Check) | value |
| ISO03 | V3 | 19.0 | 4.1 | 0.434 | −0.331 | 0.063 |
| ISO07 | V3 | 28.6 | 9.9 | 0.484 | −0.281 | 0.126 |
| ISO02 | V3 | 22.8 | 7.2 | 0.489 | −0.276 | 0.148 |
| ISO01 | V3 | 27.4 | 13.3 | 0.514 | −0.251 | 0.212 |
| ISO09 | V3 | 26.3 | 18.9 | 0.521 | −0.244 | 0.170 |
| ISO04 | V3 | 30.7 | 9.7 | 0.541 | −0.224 | 0.220 |
| ISO10 | V3 | 28.8 | 10.0 | 0.541 | −0.224 | 0.207 |
| ISO12 | V3 | 33.4 | 12.4 | 0.569 | −0.196 | 0.286 |
| ISO05 | V3 | 40.0 | 11.0 | 0.668 | −0.097 | 0.583 |
| ISO11 | V3 | 38.9 | 11.7 | 0.669 | −0.096 | 0.599 |
| Check | V3 | 52.5 | 12.8 | 0.765 | 0.000 | — |
| ISO08 | V3 | 49.2 | 9.6 | 0.781 | 0.016 | 0.931 |
| ISO13 | V3 | 50.9 | 10.9 | 0.785 | 0.020 | 0.908 |
| ISO06 | V3 | 50.0 | 12.1 | 0.808 | 0.043 | 0.811 |
| ISO14 | V3 | 63.3 | 10.4 | 0.933 | 0.168 | 0.376 |
| ISO07 | R1 | 27.9 | 12.1 | 0.497 | −0.100 | 0.581 |
| ISO10 | R1 | 29.0 | 10.1 | 0.498 | −0.099 | 0.576 |
| ISO08 | R1 | 30.0 | 10.6 | 0.550 | −0.047 | 0.803 |
| ISO09 | R1 | 31.3 | 9.2 | 0.568 | −0.029 | 0.870 |
| ISO02 | R1 | 27.5 | 9.6 | 0.569 | −0.028 | 0.882 |
| ISO12 | R1 | 33.1 | 12.3 | 0.569 | −0.028 | 0.878 |
| ISO13 | R1 | 35.6 | 12.8 | 0.596 | −0.001 | 0.993 |
| Check | R1 | 35.8 | 13.7 | 0.597 | 0.000 | — |
| ISO05 | R1 | 34.4 | 10.2 | 0.605 | 0.008 | 0.968 |
| ISO04 | R1 | 38.4 | 11.8 | 0.633 | 0.036 | 0.846 |
| ISO03 | R1 | 44.4 | 11.4 | 0.692 | 0.095 | 0.594 |
| ISO11 | R1 | 40.7 | 11.4 | 0.696 | 0.099 | 0.588 |
| ISO01 | R1 | 43.0 | 8.5 | 0.725 | 0.128 | 0.525 |
| ISO06 | R1 | 50.0 | 12.3 | 0.787 | 0.190 | 0.300 |
| ISO14 | R1 | 75.0 | 7.4 | 1.057 | 0.460 | 0.017 |

In the analysis conducted on plot yields for each treatment, IS002 applied at the V3 stage had a significant (p=0.2) yield increase over the check (Table 5).

TABLE 5
Effect of fourteen PPFM isolates on corn yield

| PPFM | | Mean Yield | Δ (Isolate − | |
|---|---|---|---|---|
| Isolate | Stage | Bu/Acre | Check) | P value |
| ISO02 | V3 | 80.926 | 15.564 | 0.250 |
| ISO03 | V3 | 78.717 | 13.355 | 0.307 |
| ISO04 | V3 | 77.373 | 12.011 | 0.345 |
| ISO07 | V3 | 69.799 | 4.436 | 0.734 |
| ISO01 | V3 | 69.094 | 3.732 | 0.784 |
| ISO12 | V3 | 67.159 | 1.796 | 0.891 |
| Check | V3 | 65.362 | — | — |
| ISO10 | V3 | 65.347 | −0.015 | 0.999 |
| ISO08 | V3 | 64.507 | −0.855 | 0.948 |
| ISO05 | V3 | 62.998 | −2.364 | 0.852 |
| ISO13 | V3 | 61.054 | −4.308 | 0.734 |
| ISO11 | V3 | 58.910 | −6.452 | 0.611 |
| ISO09 | V3 | 58.291 | −7.071 | 0.578 |
| ISO14 | V3 | 55.347 | −10.015 | 0.445 |
| ISO06 | V3 | 53.533 | −11.829 | 0.367 |
| ISO07 | R1 | 71.968 | 7.974 | 0.530 |
| ISO05 | R1 | 71.475 | 7.482 | 0.556 |
| ISO02 | R1 | 70.717 | 6.724 | 0.596 |
| ISO08 | R1 | 69.364 | 5.371 | 0.691 |
| ISO13 | R1 | 67.563 | 3.569 | 0.779 |
| ISO03 | R1 | 66.214 | 2.220 | 0.861 |
| ISO09 | R1 | 64.878 | 0.885 | 0.944 |
| ISO01 | R1 | 64.429 | 0.435 | 0.976 |
| Check | R1 | 63.994 | — | — |
| ISO04 | R1 | 59.695 | −4.299 | 0.741 |
| ISO11 | R1 | 59.491 | −4.503 | 0.730 |
| ISO12 | R1 | 58.335 | −5.659 | 0.666 |
| ISO10 | R1 | 57.868 | −6.126 | 0.629 |
| ISO06 | R1 | 49.734 | −14.259 | 0.277 |
| ISO14 | R1 | 41.982 | −22.012 | 0.106 |

There was a range in the degree of response to corn rootworm feeding across the 14 isolates tested. Isolates ISO02, IS003 and IS004 exhibited a reduction in root lodging and also increased corn yields in comparison to the untreated checks, which are indicative of reductions in CRW damage to the plants treated with those isolates. Isolates ISO11, ISO09, ISO14, and IS006 appear to be CRW inactive Methylobacterium sp. in so far as they did not provide for significant reductions in reductions in CRW damage when compared to a check. Treatment with ISO14 appeared to result in increased CRW damage in comparison to untreated control plants in both lodging and yield tests.

### Example 2. Identification of nucleic acid polymorphisms present in CRW-active Methylobacterium

The PPFM strains listed in Table 1 were grown on solid agar media comprising Ammonium Mineral Salts (AMS) plus glycerol and peptone at 30° C. for 5 days, essentially as described in co-assigned U.S. Patent Application Publication No. US20130324407 and incorporated herein by reference in its entirety. Genomic DNA was extracted using MO-BIG (Carlsbad, CA) Ultra Clean Microbial DNA Isolation kit, and 1 ug of high quality DNA was used for Illumina Nextera XT library preparation followed by Illumina 2×100 paired-end sequencing on a HiSeq2000 system. Raw Illumina genomic sequence data were subjected to adaptor- and quality-based trimming for quality control. Whole-genome Shotgun Sequence Assembly was achieved by assembling quality-passed data using the de novo assembler SPADES (33). For gene finding and annotation, reference training data was leveraged from TIGRFAM (9), Pfam, COG (10), and UniRefl00 (11). The rRNAs were identified with RNAmmer (5), protein-coding genes were identified with Glimmer (3) and Maker (6), and tRNAs were identified with tRNAscan-SE (4). Gene functions were assigned with blastx (7), blastp (7), HMMER (8), and InterProScan against comprehensive protein databases described above (Reference Data). Detection of polymorphisms (SNP or other DNA variations occurring as a result of insertions, deletions, and substitutions (Indels)) in the Methylobacterium sp. isolates ISO01-ISO14 was performed with BWA (12) and the Samtools suite (on the internet at samtools.sourceforge.net/) and the Genome Analysis Toolkit (GATK, on the world wide web internet site “broadinstitute.org/gatk/”), structural variation was identified with BreakDancer (on the internet at breakdancer.sourceforge.net/) and CoGE (on the internet at genomevolution.org/CoGe/).

### Example 3. Identification of Polymorphisms

Polymorphisms found in CRW-active Methylobacterium sp. will be identified by comparisons of the sequences of CRW-active Methylobacterium sp. ISO01, ISO02, ISO03, ISO04, and/or IS007 to Methylobacterium sp. ISO010, ISO08, ISO05, ISO13, ISO11, ISO09, ISO14, and IS006 that do not exhibit CRW activity. Nucleic acid polymorphisms present in CRW-active Methylobacterium sp. ISO01, ISO02, ISO03, ISO04, and/or IS007 that were absent in Methylobacterium sp. ISO010, ISO08, ISO05, ISO13, ISO11, ISO09, ISO14, and IS006 that do not exhibit CRW activity will be used to identify other candidate Methylobacterium sp. to test for CRW-activity. In certain embodiments, nucleic acid polymorphisms present in CRW-active Methylobacterium sp. ISO01, ISO02, ISO03, ISO04, and/or IS007 that were absent in Methylobacterium sp. ISO14, a strain which appeared to result in increased CRW damage in comparison to untreated control plants, will then be used to identify other candidate CRW-active Methylobacterium sp. to test for CRW-activity.

### Example 4. Treatment of Seedlings with Methylobacterium sp.

The fourteen PPFM strains ISO1-ISO14 were tested in the following manner. Bacterial cultures at a titer of 1×10E7 to 1×10E9 colony forming units/milliliter were used to coat 3-day-old sterile corn seedlings (germinated at 28 degrees) in sterilized soil mix in sundae cups, and the plants were watered and covered with a lid. The following day, 10 corn rootworm (CRW) larvae (1st instars) were transferred to each cup. Seedlings were grown at 25 degrees centigrade in a growth chamber for 14 additional days (18 days total) before harvest. The soil component containing roots and larvae were placed in Berlese funnels to collect live larvae. Each experiment included 3 reps each of controls and samples both with and without CRW larvae, and the experiment was repeated 4×. Data collected included shoot and root dry weights as well as number and size of larvae. There were no statistically significant differences between PPFM-treated plants and controls at p<0.05 in shoot and root dry weights. There were also no statistically significant differences in the number and size of recovered larvae from PPFM-treated plants and controls at p<0.05.

### Example 5. Evaluation of Additional Methylobacterium strains

An experiment was conducted as a 2-factorial randomized complete block (RCB), with 8 Methylobacterium strains, 2 controls (media and dead powder) and 2 corn types, Viking 5291 and CRW3(S1)C6 (a germplasm with resistance to CRW), with 4 replicates per treatment. Corn seeds were washed and incubated at 25° C. overnight in purified water to speed germination. Seeds were dried and treated with Methylobacterium strains at a rate of 1×10⁶ CFU per seed. UV lights were used for 10 minutes to surface sterilize all materials used. On day 1, three seeds per treatment were planted in a 50 mL tube (˜2 cm in depth) containing 45 ml of growing media and 15 mL of water. The growing medium consisted of 2:1 soil:Promix (peat based, particle size: through a standard 30 mesh sieve (600 μm, but not through 80 mesh (180 μm)) that was autoclaved twice. Plants were grown in a growth chamber maintained at 20° C., ˜60% RH, and a photoperiod of 14:10 (L:D) h. On day 5, eight corn rootworm neonates (<24 h old) were transferred to each tube using a fine paintbrush. The temperature was increased to 25° C. in the growth chamber. Larvae were allowed to feed on plants for seven days. On day 12, tubes were placed in Berlese funnels for ˜36 hours to collect larvae and corn roots. Larvae were collected into 90% ethanol, dried in an oven for 48 h at 55° C., and weighed. Larval dried weight and mortality of corn roots were recorded and analyzed.

Although Methylobacterium treatments were not significantly different in this study, variations between strains were observed (Table 6). Higher percent mortality was observed when larvae were reared on Viking corn line compared with the CRW3(S1)C6.

TABLE 6

| | Viking 5291 | CRW3(S1) C6 |
|---|---|---|
| Treatment | % larval mortality | % larval mortality |
| NLS0042 Betaine | 89.58 | 76.04 |
| NLS0038 Glycerol stock | 82.29 | 81.25 |
| UTC | 83.33 | 77.08 |
| NLS0042 Methanol | 86.46 | 73.96 |
| NLS0020 Glycerol stock | 86.46 | 85.42 |
| Dead powder | 88.54 | 80.21 |
| NLSCRW11 Glycerol stock | 75.00 | 73.96 |
| NLS0037 Glycerol stock | 85.42 | 73.96 |
| NLSCRW8 powder | 73.96 | 80.21 |
| NLS0069 Glycerol stock | 83.33 | 78.13 |
| NLS0617 Glycerol stock | 87.50 | 71.88 |
| Freezer media | 88.54 | 78.13 |
| NLS0020 Powder | 85.42 | 81.25 |
| NLS0042 Glycerol stock | 85.42 | 79.17 |

### REFERENCES

1. Miller J R, Koren S, Sutton G (2010) Assembly algorithms for next-generation sequencing data. Genomics 95: 315-327.
2. Zerbino D R, Birney E (2008) Velvet: algorithms for de novo short read assembly using de Bruijn graphs. Genome Res 18: 821-829.
3. Delcher A L, Bratke K A, Powers E C, Salzberg S L (2007) Identifying bacterial genes and endosymbiont DNA with Glimmer. Bioinformatics 23: 673-679.
4. Lowe T M, Eddy S R (1997) tRNAscan-SE: a program for improved detection of transfer RNA genes in genomic sequence. Nucleic Acids Res 25: 955-964.
5. Lagesen K, Hallin P, Rodland E A, Staerfeldt H H, Rognes T, et al. (2007) RNAmmer: consistent and rapid annotation of ribosomal RNA genes. Nucleic Acids Res 35: 3100-3108.
6. Cantarel B, Korf I, Robb S, et al. (2008) MAKER: An easy-to-use annotation pipeline designed for emerging model organism genomes. Genome Research 18: 188-196.
7. Altschul S F, Madden T L, Schaffer A A, Zhang J, Zhang Z, et al. (1997) Gapped BLAST and PSI-BLAST: a new generation of protein database search programs. Nucleic Acids Res 25: 3389-3402.
8. Eddy SR (2009) A new generation of homology search tools based on probabilistic inference. Genome Inform 23: 205-211.
9. Haft D H, Selengut J D, White O (2003) The TIGRFAMs database of protein families. Nucleic Acids Res 31: 371-373.
10. Tatusov R L, Fedorova N D, Jackson J D, Jacobs A R, Kiryutin B, et al. (2003) The COG database: an updated version includes eukaryotes. BMC Bioinformatics 4: 41.
11. Suzek B E, Huang H, McGarvey P, Mazumder R, Wu C H (2007) UniRef: comprehensive and non-redundant UniProt reference clusters. Bioinformatics 23: 1282-1288.
12. Li H. and Durbin R. (2009) Fast and accurate short read alignment with Burrows-Wheeler Transform. Bioinformatics, 25:1754-60.
13. Abanda-Nkpwatt, D., M. Musch, J. Tschiersch, M. Boettner, and W. Schwab. 2006. Molecular interaction between Methylobacterium extorquens and seedlings: growth promotion, methanol consumption, and localization of the methanol emission site. J. Exp. Bot. 57: 4025-4032.
14. Broekaert W F, Terras F R, Cammue B P, Vanderleyden J (1990) An automated quantitative assay for fungal growth inhibition. FEMS Microbiology Letters 69: 55-60.
15. Cao, Y-R, Wang, Q., Jin, R-X., Tang, S-K., He, W-X., Lai, H-X, Xu, L-H., and C-L Jiang. 2011. Methylobacterium soli sp. nov. a methanol-utilizing bacterium isolated from the forest soil. Antonie van Leeuwenhoek (2011) 99:629-634.
16. Corpe, W. A., and D. V. Basile. 1982. Methanol-utilizing bacteria associated with green plants. Devel. Industr. Microbiol. 23: 483-493.
17. Corpe, W. A., and S. Rheem. 1989. Ecology of the methylotrophic bacteria on living leaf surfaces. FEMS Microbiol. Ecol. 62: 243-250.
18. Green, P. N. 2005. Methylobacterium. In Brenner, D. J., N. R. Krieg, and J. T. Staley (eds.). “Bergey's Manual of Systematic Bacteriology. Volume two, The Proteobacteria. Part C, The alpha-, beta-, delta-, and epsilonproteobacteria.” Second edition. Springer, New York. Pages 567-571.
19. Green, P. N. 2006. Methylobacterium. In Dworkin, M., S. Falkow, E. Rosenberg, K. -H. Schleifer, and E. Stackebrandt (eds.). “The Prokaryotes. A Handbook on the Biology of Bacteria. Volume 5. Proteobacteria: Alpha and Beta Subclasses.” Third edition. Springer, New York. Pages 257-265.
20. Holland, M. A. 1997. Methylobacterium and plants. Recent. Res. Devel. in Plant Physiol. 1: 207-213.
21. Holland, M. A., and J. C. Polacco. 1994. PPFMs and other covert contaminants: Is there more to plant physiology than just plant? Annu. Rev. Plant Physiol. Plant Mol. Biol. 45: 197-209.
22. Kutschera, U. 2007. Plant-associated methylobacteria as co-evolved phytosymbionts. A hypothesis. Plant Signal Behav. 2: 74-78.
23. Lidstrom, M. E. 2006. Aerobic methylotrophic prokaryotes. In Dworkin, M., S. Falkow, E. Rosenberg, K. -H. Schleifer, and E. Stackebrandt (eds.). “The Prokaryotes. A Handbook on the Biology of Bacteria. Volume 2. Ecophysiology and biochemistry.” Third edition. Springer, New York. Pages 618-634.
24. Madhaiyan, M., S. Poonguzhali, H. S. Lee, K. Hari, S. P. Sundaram, and T. M. Sa. 2005. Pink-pigmented facultative methylotrophic bacteria accelerate germination, growth and yield of sugarcane clone Co86032 (Saccharum officinarum L.) Biol. Fertil. Soils 41: 350-358.
25. Madhaiyan, M., S. Poonguzhali, M. Senthilkumar, S. Seshadri, H. Chung, J. Yang, S. Sundaram, and T. Sa. 2004. Growth promotion and induction of systemic resistance in rice cultivar CO-47 (Oryza sativa L.) by Methylobacterium spp. Bot. Bull. Acad. Sin. 45: 315-324.
26. Madhaiyan, M., S. Poonguzhali, and T. Sa. 2007. Influence of plant species and environmental conditions on epiphytic and endophytic pink-pigmented facultative methylotrophic bacterial populations associated with field-grown rice cultivars. J Microbiol Biotechnol. 2007 October;17(10):1645-54.
27. Stanier, R. Y., N. J. Palleroni, and M. Doudoroff. 1966. The aerobic pseudomonads: A taxonomic study. J. Gen. Microbiol. 43: 159-271.
28 Sy, A., Giraud, E., Jourand, P., Garcia, N., Willems, A., De Lajudie,P., Prin, Y., Neyra, M., Gillis, M., Boivin-Masson,C., and Dreyfus, B. 2001. MethylotrophicMethylobacterium Bacteria Nodulate and Fix Nitrogen in Symbiosis with Legumes. Jour. Bacteriol. 183(1):214-220,
29. Sy, A., A. C. J. Timmers, C. Knief, and J. A. Vorholt. 2005. Methylotrophic metabolism is advantageous for Methylobacterium extorquens during colonization of Medicago truncatula under competitive conditions. Appl. Environ. Microbiol. 71: 7245-7252.
30. Vogel, H. J., and D. M. Bonner. 1956. Acetylornithinase of Escherichia coli: Partial purification and some properties. J. Biol. Chem. 218: 97-106.
31. Vogel, H. J. 1956. A convenient growth medium for Neurospora (Medium N). Microbial Genet Bull 13: 42-43
32. Whittenbury, R., S. L. Davies, and J. F. Wilkinson. 1970. Enrichment, isolation and some properties of methane-utilizing bacteria. J. Gen. Microbiol. 61: 205-218.
33. Bankevich A, Nurk S, Antipov D, Gurevich A A, Dvorkin M, Kulikov A S, Lesin V M, Nikolenko S I, Pham S, Prjibelski A D, Pyshkin A V, Sirotkin A V, Vyahhi N, Tesler G, Alekseyev M A, Pevzner P A. SPAdes: a new genome assembly algorithm and its applications to single-cell sequencing. J Comput Biol. 2012 May; 19(5):455-77. doi: 10.1089/cmb.2012.0021. Epub 2012 Apr 16. PMID: 22506599

Having illustrated and described the principles of the present disclosure, it should be apparent to persons skilled in the art that the disclosure can be modified in arrangement and detail without departing from such principles.

Although the materials and methods of this disclosure have been described in terms of various embodiments and illustrative examples, it will be apparent to those of skill in the art that variations can be applied to the materials and methods described herein without departing from the concept, spirit and scope of the disclosure. All such similar substitutes and modifications apparent to those skilled in the art are deemed to be within the spirit, scope and concept of the disclosure as defined by the appended claims.

1. A method for reducing corn rootworm (CRW) mediated damage to a corn plant that comprises: (i) applying a composition comprising a CRW-active Methylobacterium sp. selected from the group consisting of IS013 (NRRL B-50941), IS008 (NRRL B-50936), NLS0210 and NLS0617 to a corn plant, a part thereof, or a corn seed; and, (ii) growing the corn plant or a corn plant from the corn seed in the presence of CRW, thereby reducing CRW damage to the corn plant or to the corn plant grown from the corn seed.

2. The method of claim 1, wherein CRW mediated damage sustained by any of the corn plants grown in the presence of the CRW is reduced in comparison to CRW mediated damage sustained by a control corn plant grown in the presence of the CRW.

3. (canceled)

4. The method of claim 1, wherein the composition comprises the CRW-active Methylobacterium sp. at a titer of at least 1×10⁶ colony-forming units per ml for a liquid composition or at least 1×10⁶ colony-forming units per gram for a solid composition.

5. The method of claim 1, wherein the CRW-active Methylobacterium sp. has at least one polymorphic DNA element that is present in at least one CRW-active Methylobacterium sp. strain selected from the group consisting of NLSO210 and NLS0617.

6. The method of claim 1, wherein the CRW-active Methylobacterium sp. is selected from the group consisting of IS013 and IS008.

7. The method of claim 1, wherein the applied composition coats or partially coats the corn plant, the part thereof, or the corn seed.

8-10. (canceled)

11. A corn plant or corn plant part that is coated or partially coated with a composition of claim 39.

12. (canceled)

13. The corn plant or corn plant part of claim 11, wherein the composition comprises the CRW-active Methylobacterium sp. at a titer of at least 1×10⁶ colony-forming units per ml for a liquid composition or at least 1×10⁶ colony-forming units per gram for a solid composition.

14-15. (canceled)

16. The corn plant or corn plant part of claim 11, wherein the corn plant part is selected from the group consisting of a coleoptile, leaf, a stalk, and a seed.

17. A method for controlling corn rootworm (CRW) damage to a corn plant that comprises: (i) applying a composition comprising a CRW-active Methylobacterium sp. selected from the group consisting of IS013, IS008, NLSO210 and NLS0617 to soil where a corn plant is growing or will be grown, wherein the composition is applied during the growing season; and,
(ii) growing a corn plant or a corn plant from corn seed in soil subjected to the application of the composition and in the presence of CRW.

18-21. (canceled)

22. The method of claim 17, wherein the composition is applied to the soil by broadcasting the composition, by drenching the soil with the composition, and/or by depositing the composition in furrow.

23. The method of claim 22, wherein the depositing in furrow is performed prior to placing corn seed in the furrow, at the same time as placing corn seed in the furrow, or after placing corn seed in the furrow.

24. A method for treating a corn plant seed that can provide a corn rootworm (CRW) tolerant corn plant that comprises applying a composition of claim 39, to a corn seed, thereby obtaining a treated seed that can provide a CRW tolerant corn plant.

25-30. (canceled)

31. A treated corn seed obtained by the method of claim 24.

32. A method for controlling corn rootworm (CRW) damage to a corn plant that comprises: (i) planting a corn seed that has been treated with a composition comprising a CRW-active Methylobacterium sp. selected from the group consisting of ISO13, ISO08, NLSO210 and NLS0617; and,
(ii) growing a CRW-tolerant corn plant from the treated corn seed in the presence of CRW.

33-38. (canceled)

39. A composition comprising at least one CRW-active Methylobacterium sp. selected from NLSO210 and NLS0617, and an agriculturally acceptable adjuvant and/or an agriculturally acceptable excipient.

40. (canceled)

41. The composition of claim 39, wherein the composition comprises the CRW-active Methylobacterium sp. at a titer of at least 1×10⁶ colony-forming units per ml for a liquid composition or at least 1×10⁶ colony-forming units per gram for a solid composition.

42-43. (canceled)

44. The composition of claim 39, wherein the composition further comprises an insecticide that provides for inhibition of CRW growth and/or reductions in CRW-mediated plant damage.

45. The composition of claim 44, wherein the insecticide is selected from the group consisting of a pyrethrin, synthetic pyrethroid, oxadiazine, chloronicotinyl, neonicotinoid, nitroguanidine insecticide, triazole, organophosphate, pyrrol, pyrazole, diacylhydrazine, biological/fermentation product, and a carbamate.

46. The method of claim 1, wherein the composition further comprises an insecticide that provides for inhibition of CRW growth and/or reductions in CRW-mediated plant damage.

47. The method of claim 46, wherein the insecticide is selected from the group consisting of a pyrethrin, synthetic pyrethroid, oxadiazine, chloronicotinyl, neonicotinoid, nitroguanidine insecticide, triazole, organophosphate, pyrrol, pyrazole, diacylhydrazine, biological/fermentation product, and a carbamate.

48. (canceled)

49. The method of claim 23, wherein the composition is applied to the seed and soil with a planter.

50. (canceled)
